(19) United States
(12) Patent Application Publication
(10) Pub. No.: US 2009/0078326 A1
(43) Pub. Date: Mar. 26, 2009

(54) LIGHT-DRIVEN MICROFLUIDIC DEVICES AND AMPLIFICATION OF STIMULUS-INDUCED WETTING

(75) Inventors: Rohit Rosario, Mountain View, CA (US); S.Thomas Picraux, Albuquerque, NM (US); Mark Hayes, Gilbert, AZ (US); John Devens Gust, JR., Mesa, AZ (US); Antonio A. Garcia, Chandler, CA (US)

Correspondence Address: FULBRIGHT & JAWORSKI L.L.P., 600 CONGRESS AVE., SUITE 2400, AUSTIN, TX, 78701, US

(73) Assignee: ARIZONA BOARD OF REGENTS FOR AND ON BEHALF OF ARIZONA STATE UNIVERSITY, Tempe, AZ (US)

(21) Appl. No.: 11/629,365

(22) PCT Filed: Jun. 14, 2005
(86) PCT No.: PCT/US2005/020959

§ 371 Date: Dec. 2, 2008

Related U.S. Application Data
(60) Provisional application No. 60/579,827, filed on Jun. 14, 2004.

(51) Int. Cl. B81B 7/02; B32B 3/30; C23C 16/44; B05D 3/10; C12M 3/00

(52) U.S. Cl. 137/827; 428/172; 427/248.1; 427/299; 422/100; 137/806; 137/833

(57) ABSTRACT

Disclosed are compositions and methods for producing movement of liquid across surfaces.

This application claims benefit of U.S. Provisional Application No. 60/579,827, filed Jun. 14, 2004, which is hereby incorporated herein by reference in its entirety.

### ACKNOWLEDGEMENTS

This invention was made with government support under Grants CTS-0102680 and CHE-0352599 awarded by the National Science Foundation. The government has certain rights in the invention.

### BACKGROUND

The physics of scale require that microfluidic devices exploit new approaches to fluid movement because of an inherently large ratio of liquid surface area to volume. One promising method is manipulation of the water wettability of surfaces by light. See, e.g., Ichimura, K., Oh, S. & Nakagawa, M., Light-driven motion of liquids on a photoresponsive surface, Science 288, 1624-1626 (2000); Rosario, R. et al., Photon-modulated wettability changes on spiropyran-coated surfaces, Langmuir 18, 8062-8069 (2002). Light fluxes can be easily imposed, accurately controlled, and mild enough to pose no danger to biological materials. Such wettability changes are predicted to result in water drop motion if the light induces a contact angle at the advancing edge that is below that of the receding edge. However, moving water with light has not been possible previously because contact angle hysteresis (the difference between advancing and receding contact angles) is larger than the light-induced contact angle change, preventing the criterion of producing a contact angle at the advancing edge that is below that of the receding edge, from being met.

Disclosed herein are solutions to this problem that in some embodiments, are based on materials that mimic biological surfaces such as lotus leaves, which use high microscopic roughness and hydrophobic molecular coatings to produce non-wettable superhydrophobic surfaces. In one embodiment, the use of a rough, hydrophobic, photoresponsive surface, produces a sufficient amplification of contact angle switching for liquid movement. Decreased contact angle hysteresis is also disclosed. The combination of amplified contact angle switching and decreased contact angle hysteresis allows drops of water to be moved about solely under the influence of light, opening the door to enhancement of liquid motion in other surface-tension driven microfluidic systems.

### SUMMARY

Disclosed are devices, methods, and compositions related to the movement of liquid in microfluidics devices.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows an example of how the selection of a particular rough surface can increase the light-induced contact angle change. Contact angles as a function of a, feature width, and b, inter-feature distance, under both Wenzel and Cassie models, are shown.

FIG. 2 shows SEM images of nanowires as function of VLS growth time (1, 3, and 8 minutes) on a silicon surface seeded with gold nanodots. Upper panels are for plan view (scale markers 0.5 μm) and lower panels are for cross section view (scale markers 1.0 μm). After 8 minutes of growth a dense array of randomly oriented, long and thin silicon nanowires with gold caps is evident.

FIG. 3 shows advancing contact angle changes on smooth (lower) and rough photoresponsive (upper) surfaces under UV and visible light irradiation. Smooth and rough surface measurements are represented by triangles and squares, respectively. Average contact angles on smooth and rough surfaces are given by black and grey lines, respectively. The dashed line shows the predicted rough contact angle using the Wenzel model for fractally rough surfaces.

FIG. 4 shows a schematic representation of a drop of liquid sitting on a fractally rough composite surface made up of solid and air.

FIG. 5 shows an example from a class of organic photochromes, known generally as Spiropyrans, that undergo a reversible transition from a closed, nonpolar, form to a highly polar, open form when irradiated with higher energy, shorter wavelength light (e.g., ultraviolet (UV) light).

FIG. 6 shows an example of a class of organic photochromes, known generally as Dihydroindolizines, that undergo a reversible transition from a closed, nonpolar, form to a highly polar, open form when irradiated with higher energy, shorter wavelength light (e.g., ultraviolet (UV) light).

FIG. 7 shows an example of a class of organic photochromes, known generally as Dithienylethenes, that undergo a reversible transition from an open, nonplanar form to a closed, planar form when irradiated with higher energy, shorter wavelength light (e.g., ultraviolet (UV) light).

FIG. 8 shows an example of a class of organic photochromes, known generally as Dihydropyrenes, that undergo a reversible transition from a closed, planar form to an open, nonplanar form when irradiated with higher energy, shorter wavelength light (e.g., ultraviolet (TV) light).

FIG. 9 shows a cross-sectional SEM of air-oxidized Si nanowires on a Si substrate.

FIG. 10 shows examples of water drops on surfaces having identical spiropyran coatings and irradiated with visible light. Smooth surface (left) and rough nanowire surface (right).

FIG. 11 shows advancing (squares) and receding (triangles) contact angles for nanowire surfaces as a function of the advancing contact angle for a sample with similar surface chemistry on a smooth surface. The solid line is the contact angle predicted by the Cassie-Baxter equation with constant value of f, and the dashed line is the contact angle predicted by the Wenzel model.

FIG. 12 shows the predicted effect of fractal dimension on a 108°-95° light-induced contact angle change. Limits of fractal behavior used in this example are l=0.2 μm and L=36 μm. The lower graph shows the difference between the upper and middle graphs.

FIG. 13 shows an experimental setup that can be used according to Example 1.

FIG. 14 shows a spectrum of visible light emitted from a FiberOptic Specialities lamp (left), and a spectrum of ultraviolet light emitted from an Ocean Optics UV lamp (right).

FIG. 15 shows a cross sectional view of a water droplet on a fractally rough hydrophobic surface as treated with a 1 mm beam of ultraviolet light.

FIG. 16 shows a cross sectional view of a water droplet on a fractally rough hydrophobic surface as treated with a 1 mm beam of ultraviolet light.

FIG. 17 shows a cross sectional view of a water droplet on a fractally rough hydrophobic surface in a control experiment without treatment with ultraviolet light.

FIG. 18 shows a schematic representation of droplet-surface interaction according to the Cassie model.

FIG. 19 shows a schematic representation of droplet-surface interaction according to the Wenzel model.

FIG. 20 shows a schematic representation of an exemplary photoresponsive hydrophobic surface as disclosed herein.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Devices

1. Microfluidics

Microfluidic devices are, essentially, tiny, sophisticated devices that can analyze samples. Continuous flow systems have generally been the default approach towards such lab-on-chip bioassay systems. Fluid droplet based lab-on-chip applications, however, have become increasingly popular because of their ability to enable spatially and temporally resolved chemistries.

Typical microfluidic devices can have one or more channels with at least one dimension less than 1 mm and can be used with common fluids including, for example, whole blood samples, bacterial cell suspensions, protein or antibody solutions, and various buffers.

Molecular diffusion coefficients, fluid viscosity, pH, chemical binding coefficients, and enzyme reaction kinetics can be measured by using microfluidic devices. Microfluidic devices can also be used in many applications relating to clinical diagnostics, for example, capillary electrophoresis, isoelectric focusing, immunoassays, flow cytometry, sample injection of proteins for analysis via mass spectrometry, polymerase chain reaction (PCR) amplification, DNA analysis, cell manipulation, cell separation, cell patterning, and chemical gradient formation.

For example, in a microfluidic device, the cells, DNA, or proteins that are used to test the candidate drug efficacy can be reduced so that a small amount of a candidate drug can be mixed with its target and the result recorded. This can reduce the time needed to screen all of the drug candidates and can allow as many tests as possible to be run simultaneously. For example, a microfluidic device can require only a single drop of blood for a battery of twenty to thirty tests and can provide nearly immediate results. Microfluidic devices can also help pharmaceutical companies, for example, screen for new drugs by allowing tests to be run on an extremely small scale and in a simultaneous fashion.

The small size and parallel nature of microfluidic devices can create significant advantages. First, because the volume of fluids within these channels is very small, usually only several nanoliters, the amounts of reagents and analytes used are quite small, compared with traditional analysis methods. Second, fabrication techniques used to construct microfluidic devices can be relatively inexpensive and are compatible with elaborate, multiplexed devices and with mass production. Third, microfluidic devices can be fabricated as highly integrated devices for performing a plurality of functions on the same substrate chip.

Fluids are typically driven through microfluidic devices by either pressure driven flow or by electro-osmotic pumping. In pressure driven flow, the fluid can be pushed through the device by using a positive displacement pump, for example, a syringe pump. Pressure driven flow can be both relative inexpensive and quite reproducible. Pressure driven flow can be useful for continuous flow systems but is less useful for fluid droplet based lab-on-chip applications. In electro-osmotic pumping, an electric field can be applied across the microchannels of the microfluidic device. Ions in the surface of the walls of the microchannels move towards the electrode of opposite polarity, resulting in motion of the fluid near the walls and transfers via viscous forces into convective motion of the bulk fluid. Electro-osmotic pumping can be useful for both continuous flow systems and for fluid droplet based lab-on-chip applications.

Other pumping devices that can be used with microfluidic devices include, without limitation, Mechanical Micropumps, such as centrifugal pumps (CD technology), peristaltic pumps, reciprocating pumps, rotary pumps, sonic pumps, ultrasonic pumps, surface acoustic wave (SAW) pumps and Nonmechanical Micropumps, such as capillary pumps, thermocapillary micropumps, electrocapillary (electrowetting) micropumps, electro-hydro dynamic (EHD) pumps, EHD static pumps (EHD injection pumps), EHD dynamic pumps (traveling or EHD induction pumps), electrokinetic pumps, electro-osmotic pumps, electrophoretic pumps, magneto-hydro dynamic (MHD) pumps, and dielectrophoretic pumps.

It is understood that the inducible microfluidic devices, as disclosed herein, can be used in combination with any other type of microfluidic device or method, as discussed herein for example.

Microfluidic devices have a variety of applications including, without limitation, chemical microplants, lab-on-a-chip (LOC) devices, micro total analysis systems (μTAS), microfactories, microseparation systems, and point-of-care (POC) devices.

Chemical microplants are miniaturized chemical plants. A chemical microplant is generally best suited for a distributed processing of materials at the point-of-use. Such distributed processing could avoid central storage and transportation of toxic substances. Another application could be for substances that are needed only in small quantities.

Lab-on-a-chip (LOC) devices are small chips containing microfluidic channels narrower than a human hair. These devices take advantage of the properties of liquids and gases to separate and better allow microsensors to analyze their constituent elements.

Micro Total Analysis Systems (μTAS) are miniaturized systems fabricated by the use of micromechanical technology capable of providing total chemical analysis on a microliter scale. The microdevice, fully integrated for example onto a silicon substrate (chip), can perform sample handling, reagent mixing, sample component separation, and analysis. A major area of interest has been the transfer of separation techniques such as capillary electrophoresis (CE) and high performance liquid chromatography (HPLC) to the chip format, coupled with detection systems such as spectrophotometric or conductometric detectors. MicroTAS can be also used in biochemistry for DNA chip analysis and drug discovery studies.

Microfactories provide micro-scaled production. This involves parallel production. Explosive reactions or reaction demanding intensive heat exchange can be divided into safer microreactions, but still providing the same volume of production.

Microseparation systems are miniaturized separation systems.

Point-of-care (POC) devices involve diagnostic testing carried out when a patient visits the clinic, with the results available at that visit. Such devices usually consist of a disposable test cartridge and a reading device, usually hand-held or desktop sized.

Microfluidic devices can be fabricated from a variety of materials. Silicon (Si) has been used extensively in microfluidic devices. Silicon can be an especially good material for microfluidic channels coupled with microelectronics or other microelectromechanical systems (MEMS). It also has good stiffness, allowing the formation of fairly rigid microstructures, which can be useful for dimensional stability. In these applications as well as in the use of silicon herein, the silicon surface is typically a silicon oxide that naturally forms upon exposure of silicon to air or that is formed by another oxidation method.

Generally, a photoresist is spun onto a silicon substrate. The photoresist is then exposed to ultraviolet (UV) light through a high-resolution mask with the desired device patterns. After removing the excess unpolymerized photoresist, the silicon wafer is placed in a wet chemical etching bath that anisotropically etches the silicon in locations not protected by photoresist, resulting in a silicon wafer in which microchannels are etched. A glass coverslip can be used to fully enclose the channels and holes are drilled in the glass to allow fluidic access. For straighter edges and a deeper etch depth, deep reactive ion etching (DRIE) is an alternative to wet chemical etching.

Another material suitable for microfluidic device is polydimethylsiloxane (PDMS). Generally, liquid PDMS is poured over a mold and cured to cross-link the polymer, resulting in an optically clear, relatively flexible material that can be stacked onto other cured polymer slabs to form complex three dimensional geometries.

2. Roughness

The disclosed devices, compositions, and methods can incorporate roughness as discussed herein to aid in the production of a more hydrophobic surface.

One approach to preparing microscopically rough surfaces has been the use of photolithographic methods. For example, standard photolithography with a resist can be used to prepare surfaces with defined surface feature (pillar arrays) dimensions in an n-type silicon substrate. The height of the surface features, h, is specified by the etch depth.

In another approach, x-ray lithography techniques, such as (S)LIGA, can be used to define high aspect ratio structures in nickel. The process consists of exposing a sheet of PMMA bonded to a wafer using X-ray lithography. The PMMA is then developed and the exposed material is removed. Nickel is then electroplated up in the open areas of the PMMA. The nickel over-plate is removed by polishing, leaving high aspect ratio nickel parts. The PMMA is removed, and the nickel parts may remain anchored to the substrate or be released.

Rough surfaces including surface features can be prepared by physical vapor deposition methods that include, for example, evaporation and sputtering.

In evaporative methods, a substrate can be placed in a high vacuum chamber at room temperature with a crucible containing the material to be deposited. A heating source can be used to heat the crucible causing the material to evaporate and condense on all exposed cool surfaces of the vacuum chamber and substrate. Typical sources of heating include, for example, e-beam, resistive heating, RF-inductive heating. The process typically can be performed on one side of the substrate at a time. In some systems, the substrate can be heated during deposition to alter the composition/stress of the deposited material.

In sputtering methods, a substrate can be placed in a vacuum chamber with a target (a cathode) of the material to be deposited. A plasma is generated in a passive source gas (e.g., Argon) in the chamber, and the ion bombardment is directed towards the target, causing material to be sputtered off the target and condense on the chamber walls and the substrate. A strong magnetic field can be used to concentrate the plasma near the target to increase the deposition rate. The ejection of atoms or groups of atoms from the surface of the cathode of a vacuum tube can be the result of heavy-ion impact. Sputtering methods can be used to deposit a thin layer of metal on a glass, plastic, metal, or other surface in a vacuum.

Chemical vapor deposition (CVD) methods can also be used to prepare rough surfaces. CVD methods pertain to the growth of thin solid films on a crystalline substrate as the result of thermochemical vapor-phase reactions. CVD methods include, for example, low-pressure chemical vapor deposition (LPCVD) and plasma enhanced chemical vapor deposition (PECVD).

LPCVD can be performed in a reactor at temperatures up to about 900° C. A deposited film is a product of a chemical reaction between the source gases supplied to the reactor. The process typically can be performed on both sides of the substrate at the same time.

PECVD can be performed in a reactor at temperatures up to about 400° C. The deposited film is a product of a chemical reaction between the source gases supplied to the reactor. A plasma is generated in the reactor to increase the energy available for the chemical reaction at a given temperature. The process typically can be performed on one side of the substrate at a time.

In the present devices, compositions, and methods, multidimensionally rough surfaces can be prepared as disclosed herein, and with the characteristics as disclosed herein.

Surface energy gradients can be designed by preparing surfaces having varying degrees of roughness. For example, chemically homogeneous surfaces of varying roughness can be prepared by photolithographic techniques. To prepare a surface roughness gradient, for example, substantially parallel strips of surfaces can be prepared and positioned so that fluid droplets in contact with the surface will contact at least two strips along the surface roughness gradient. Surface features are typically at least one order of magnitude smaller than the fluid droplet size. The strips can be selected such that each strip has a successively greater surface roughness. A path that is substantially perpendicular to the strips, therefore, constitutes a gradient of surface roughness. In such a system, the fluid droplet sequentially contacts strips of increasing roughness as it moves from strips of lower roughness to strips of greater roughness, thereby successively minimizing its contact angle with the surface as roughness increases.

3. Surface Tension Driven Microfluidic System

At the microscale, surface tension becomes a relatively large force, as compared to other forces such as gravity or structural stiffness. In mechanical devices, surface tension begins to dominate other forces when physical features are shrunk to micrometers. Electrocapillary and electrowetting actively use surface tension at the microscale. Electrowetting is an electrically-induced change of a material's wettability.

Surface tension driven microfluidic systems employ surface tension to generate motion in fluid droplets. For example, hydrophobic and hydrophilic interactions of the fluid droplet with the system surface drive the droplet from regions of greater hydrophobicity (lower hydrophilicity) to regions of lower hydrophobicity (greater hydrophilicity) along a gradient of successively decreasing hydrophobicity (increasing hydrophilicity).

Fractally rough surfaces generally provide a highly involved and intricate interface with fluid droplets in contact with the surface. The contact angle at the interface between the fractally rough surface and the fluid droplet can be high, often approaching the theoretical maximum of a 180° apparent contact angle. Accordingly, fractally rough surfaces possess a smaller level of contact angle hysteresis than well-ordered surfaces or surfaces that are rough at the microscale but not at the nanoscale.

4. Liquids

a) Ratio of Surface Area to Volume

Generally, at the microscale, for example in microfluidic devices, the ratio of surface area to volume of a given liquid is extremely high compared to the ratio of surface area to volume at normal scales. Accordingly, surface properties and interactions begin to dominate other properties and interactions.

b) Liquid Drop

The liquids as disclosed herein can be in the form of drops or droplets which represent discreet self contained units of the liquid. The drops and droplets can be any size, such as the sizes disclosed herein. The word “drop” or “droplet,” when applied to a fluid, can include any discrete portion of fluid, including a free standing drop or portion on a surface, a portion of fluid in a capillary, channel, or similar partially confined space, and fluid portions within a porous medium.

In one aspect, the fluid droplet can be a liquid in contact with a solid surface and surrounded by a gaseous fluid. The gaseous fluid can be, for example, air, oxygen, nitrogen, argon, or any other suitable gas. In this aspect, the droplet moves along the surface through the gaseous fluid in response to varying hydrophobicity initiated by photoresponse of the surface coating when exposed to ultraviolet or visible light.

In another aspect, the fluid droplet can be a liquid in contact with a solid surface and surrounded by a liquid fluid. The liquid fluid can be, for example, a hydrocarbon, a lipid, a polyalkyloxide, or biphasic water. In this aspect, the droplet moves along the surface through the liquid fluid in response, for example, to varying hydrophobicity initiated as disclosed herein of the surface coating when exposed to ultraviolet or visible light, for example. In this aspect, when the droplet is surrounded by a liquid fluid, the contact angle of the droplet with the surface can be greater than the contact angle of the droplet with the surface when the droplet is surrounded by a gaseous fluid. A greater contact angle can, therefore, decrease contact hysteresis between the droplet and the surface, thus increasing the efficiency of the droplet movement. In the biphasic water example, the droplet which is in essence floating within the biphasic water liquid has a decreased amount of contact with the surface, thus in essence increasing the hydrophobicity of the surface for the droplet.

5. Liquid Surface Interactions

a) Contact Surface

A contact surface is the surface of a device or material or composition which forms an interaction with a liquid as disclosed herein. The surface can be for example silicon as discussed or any other appropriate surface for use in the disclosed devices. It is understood that the oxide of silicon, or any other metal or oxidizable molecule, is disclosed when the molecule is disclosed and vice versa, unless expressly indicated to the contrary or would be so understood by a skilled artisan. For example, gold can be used as the surface or can be used as silicon is used herein.

b) Contact Angle

Advancing angles generally follow Cassie-Baxter wetting with a constant fraction of the surface wetted for a particular roughness, while receding angles generally follow Wenzel wetting. Due to contact with the surface, Wenzel wetting creates the condition for water drop movement. The photowetting driving force is proportional to roughness.

c) Contact Angle Hysteresis

The contact angle hysteresis is the difference between the advancing contact angle and the receding contact angle in resistance to motion of the fluid droplet. If the contact angle hysteresis is larger than the light induced contact angle change, for example, contact hysteresis occurs, and movement of the fluid is slowed or stopped.

This hysteresis effect can be caused by the interaction of the receding edge with the surface. For example, attractive interactions between the surface and the fluid at the receding edge can retard motion of the fluid droplet. Hysteresis can make the driving force smaller and hence slow the speed of movement. Hysteresis can be overcome by using very rough surfaces in combination with surface modification by hydrophobic molecules, as disclosed herein. Because at constant velocity the driving force equals the drag force and hence the smaller the drag force the lower the velocity, a small difference means a slower velocity.

(1) Reduced Contact Angle Hysteresis

Another reason for using superhydrophobic rough surfaces, as disclosed herein, in surface-tension driven microfluidic applications is that the level of contact angle hysteresis reported on these surfaces can be low. See, Lafitma, A. & Quere, D., Superhydrophobic states. Nature Materials 2, 457-460 (2003); Bico, J., Marzolin, C. & Quere, D. Pearl drops. Europhysics Letters 47, 220-226 (1999); Shin, J.-Y., Kuo, C.-W., Chert, P. & Moth C.-Y. Fabrication of tunable superhydrophobic surfaces by nanosphere lithography, Chemistry of Materials 16, 561-564 (2004). One reason for the small degree of hysteresis is the very low solid-surface free energy resulting from the hydrophobic molecular coating. See Chibowski, E., Surface free energy of a solid from contact angle hysteresis. Advances in Colloid and Interface Science 103, 149-172 (2003).

Fractally-rough surfaces are particularly interesting for microfluidic applications as there are indications that these surfaces possess a smaller level of contact angle hysteresis than well-ordered ones. See, e.g., Shin, J.-Y., Kuo, C.-W., Chert, P. & Moth C.-Y. Fabrication of tunable superhydrophobic surfaces by nanosphere lithography, Chemistry of Materials 16, 561-564 (2004); Ramos, S. M. M., Charlaix, E. & Benyagoub, A., Contact angle hysteresis on nano-structured surfaces, Surface Science 540, 355-362 (2003). This phenomenon can be due to the instability of the three-dimensional, tortuous solid-liquid-gas contact line in randomly rough surfaces as compared to that in well-ordered two-dimensional rough surfaces.

Accordingly, photoresponsive monolayer coatings on fractally rough, superhydrophobic surfaces can exhibit contact angle magnification and lowered contact angle hysteresis. Using this approach, contact angle amplification and hysteresis reduction were improved by as much as a factor of two. The light-induced movement of water drops on a surface was thus demonstrated for the first time.

In an alternative aspect, the fluid droplet can comprise a liquid other than water. For example, the fluid droplet can be a nonpolar liquid such as an oil or an organic solvent. In this aspect, the fractally rough silicon nanowire-bearing surfaces can be used as suitably rough surfaces. Likewise, the disclosed spiropyrans can be used as a photosensitive variable hydrophobicity agent in this aspect.

However, in order to minimize solid-surface free energy and interactions with the nonpolar droplet, and therefore minimize contact angle hysteresis for the nonpolar fluid droplet, a hydrophilic (polar) surface coating can be used.

Exemplary hydrophilic coating materials can include ethylene glycol, ethylene glycol derivatives, polyethylene glycol, polyethylene glycol derivatives, polyvinylpyrrolidone, polyvinylpyrrolidinone derivatives, and the like.

Hydrophilic surfaces can also be prepared by contacting silicon surfaces with diluted sulfuric acid, nitric acid, or hydrofluoric acid, thereby producing a top layer consisting of hydroxyl) moieties on the oxide surface.

In this aspect, a nonpolar fluid droplet placed upon a suitably rough surface that has been coated with a photosensitive variable hydrophobicity agent can be induced to move by exposure to an ultraviolet-visible light gradient. However, in this aspect, the nonpolar fluid droplet is induced to move in the direction of increasing hydrophobicity. That is, the droplet would move in a direction opposite to that which would be moved by a water droplet in the same ultraviolet-visible light gradient.

d) Contact Angle Switching and Contact Angle Change

It is understood that the contact angles of the liquids disclosed herein can switch or change under different conditions, such as when on different surfaces having different levels of hydrophobicity.

e) Photo Induced Changes in Water Contact Angle

Photowetting is the movement of water drops on surfaces using light. This movement can be achieved by using hydrophobic nanostructured surfaces that amplify the light-induced contact angle change while minimizing contact angle hysteresis.

f) Liquid Movement

As disclosed herein, the liquid drops on the devices have an advancing edge and a receding edge which has a particular contact angle with the surface. The advancing edge is the edge which is in the direction of movement and receding edge is the edge which is following the advancing edge.

The disclosed devices, compositions, and methods have a limit of 180 degrees as an optimum receding contact angle, which in essence is when the droplet of liquid is just a tangential point on the surface. The devices, compositions, and methods, can preferably work, however in a range from 160 to 180 degrees of the receding contact angle, such as 160, 161, 162, 163, 164, 165, 166, 167, 168, 169, 170, 171, 172, 173, 174, 175, 176, 177, 178, 179, and 180 degrees.

### C. Components

Disclosed are devices comprising a surface, wherein the surface has roughness, a hydrophobic layer, and a photoresponsive molecule. A photo responsive molecule can be, for example, a spiropyran, a dihydroindolizine, a dithienylethene, a dihydropyrene, or a mixture thereof.

Polarity changing molecules, hydrophobicity changing molecules, and molecules which affect the contact angle of a liquid in contact with the surface can also be used. A polarity changing molecule can be, for example, a spiropyran. A hydrophobicity changing molecule can also be a spiropyran. A spiropyran is also a molecule which can affect the contact angle in contact with the surface. It is understood that the combination of surface, roughness, the hydrophobic layer, and for example a photoresponsive molecule, a polarity changing molecule, and a hydrophobicity changing molecule together act to affect the contact angle of a liquid in contact with the surface as disclosed herein.

1. Rough or Roughness

The disclosed devices, compositions, and methods have a roughness associated with them as disclosed herein.

Another aspect of the certain embodiments of the disclosed devices, compositions, and methods, is that there is a combination of what can be called microscale roughness and nanoscale roughness. Microscale roughness is roughness that would be considered to be on the micron scale, whereas nanoscale roughness is considered to be roughness on the nanometer scale. For example, in certain embodiments disclosed herein, the microscale roughness is caused by the length of the nanowires that are formed, which can be microns in length, and the nanoscale roughness is formed by the diameters of the nanowires, and how they interact, but also by much smaller nanowires that are formed, for example, on the microscale wires, which may be nanometer or subnanometer in length.

With respect to the size of the roughness one characteristic is that the scale of roughness should be smaller than the drop of liquid, such as for example, by 2, 3, 4, 5, 6, 7, 8, 9, 10, 20, 30, 40, 50, 60, 70, 80, 90, 100, 200, 300, 400, 500, 600, 700, 800, 900, or a 1000, 2000, 3000, 4000, 5000, 6000, 7000, 8000, 9000, or 10000, or even greater, which can be microscale roughness. And then the nanoscale can be similar orders of magnitude smaller than this. The effect of this is to create very large surface areas relative to the drop. In one aspect the roughness is a tool for modulating the relationship between the liquid and the surface and can be considered as a continuous modulator, much like a rheostat or volume control, which can be dialed in to adjust the relationship between the drop of liquid and the surface. It is understood, as disclosed herein that this occurs in conjunction with the other characteristics and compositions disclosed herein, such as, for example, the addition of the photoresponsive molecule which can make a hydrophobic surface become superhydrophobic. In any case, the roughness involves a three-dimensional structuring of the surface and can extend from a few nanometers to many micrometers in characteristic dimensions regardless of the type or shape or structure of the three-dimensional variance.

The roughness can be caused by any means which creates protrusions or variances on the surface which makes the surface less smooth. As discussed herein this can occur at any scale, such as microscale and nanoscale. For example, nanowires as discussed herein can be used to make the surface rough, but pillars can also be used as well as, for example, etched structures. It is also understood that a combination of different types and forms of structures can also be used.

Typically under the conditions of the devices, compositions, and methods disclosed herein, two effects happen at same time upon irradiation of a portion of the surface with light of suitable wavelengths or other stimulus: first, the difference between the advancing and receding contact angle begins to shrink, and second because of roughness, hydrophobicity, and the molecule which can make the devices superhydrophobic, the hysteresis can drop down to 25 degrees and below, which allows the advancing edge of the fluid to move forward and the receding edge to be “released” moving the liquid forward.

In certain embodiments the devices have a roughness where the roughness is either a well ordered microstructure, fractal geometry, or a random fractal geometry. A well ordered microstructure is one that has a regular pattern. A fractal geometry is a geometry which has a particular type of structure that builds upon itself at ever smaller scales, for example, in the disclosed nanowires, which may be at a microscale. These nanowires themselves can have nanowires growing out of them which will be smaller at the nanoscale. A random fractal geometry is one which has smaller and smaller structure building on top of existing structure, but it is not occurring such that the structures are the same composition, but just smaller: the structures themselves can change as the size changes.

Also disclosed are devices, wherein the device comprises a rough fractal surface.

Disclosed are devices, wherein the device comprises a nanoscale structure, such as a nanowire. It is understood that any materials can be used to produce the nanoscale structures, such as silicon. For example, the nanoscale structures can be semiconducting or insulating materials such as silicon oxides, zinc oxide, silicon dioxide, titanium dioxide, tungsten oxide, tantalum oxide, for example or metals, such as iron or nickel based, or alloys. Therefore, disclosed are devices, wherein the nanowire is a silicon nanowire.

Also disclosed are devices, wherein the microscale structure or nanoscale structure, such as that produced by a nanowire is in a random array of nanowires, an ordered array of nanowires, a hierarchically patterned array of nanowires, or in a combination of each. It is understood that the surface can also have regions of microscale or nanoscale structure and regions that have little or no structure, i.e. are relatively smooth.

As discussed herein, disclosed are devices wherein the structures have any combination of any size microscale or nanoscale structure. For example, the devices can have any structure, such as a diameter of a nanowire, ranging in size from between 1 nm to 100 micrometers, 10 nm to 100 micrometers, 10 nm to 200 nm, 20 nm to 500 nm, 20 nm to 100 nm, or 20 nm to 50 m.

It is understood that the disclosed microscale and nanoscale structures can be produced using any technique. For example, disclosed are devices wherein the microscale or nanoscale structure is grown by a vapor-liquid-solid technique, by a chemical or physical vapor deposition onto patterned substrates, dry plasma of pattered substrates, wet etching of patterned substrates, or by deposition of separately fabricated nanostructured materials. Disclosed are devices wherein the nanostructure is grown by a vapor liquid solid technique. Also disclosed are devices wherein the separately fabricated nanostructured materials are nanodots or nanowires.

(1) Rough Superhydrophobic Surfaces

Superhydrophobic surfaces that combine hydrophobic molecular coatings with surface roughness are generally characterized by either well-ordered microstructures, see, e.g., Lafitma, A. & Quere, D., Superhydrophobic states. Nature Materials 2, 457-460 (2003); Bico, J., Marzolin, C. & Quere, D. Pearl drops. Europhysics Letters 47, 220-226 (1999), or by random fractal geometry, see, e.g., Onda, T., Shibuichi, S., Satoh, N. & Tsujii, K., Super-water-repellent fractal surfaces, Langmuir 12, 2125-2127 (1996); Shibuichi, S., Yamamoto, T., Onda, T. & Tsujii, K., Super water- and oil-repellent surfaces resulting from fractal structure, Journal of Colloid and Interface Science 208, 287-294 (1998). Rough fractal surfaces are particularly interesting due to the extremely high degree of roughness that they possess.

The liquid contact angle on a solid surface is a function of the interfacial energy and roughness. The dependence of the apparent solid-liquid contact angle on surface roughness in terms of flat-surface contact angle can be described by the Cassie model, see Cassie, A. B. & Baxter, S., Wettability of porous surfaces, Transactions of the Faraday Society 40, 546-551 (1944), and the Wenzel model, see Wenzel, R. N., Resistance of solid surfaces to wetting by water, Industrial and Engineering Chemistry Research 28, 988-994 (1936).

Wenzel, see Wenzel, R. N., Resistance of solid surfaces to wetting by water, Industrial and Engineering Chemistry Research, 1936, 28: p. 988-994, and Cassie, see Cassie, A. B. and Baxter, S., Wettability of porous surfaces, Transactions of the Faraday Society 1944. 40: p. 546-551, developed approaches to model the rough surface contact angle, θᵣ, using average roughness characteristics of the surface. Wenzel approached the problem by assuming that the liquid filled every part of the rough surface in the region of its contact. Cassie, on the other hand, assumed that the features on the surface would lift up the liquid in the region of contact, leading to the formation of a composite surface.

Both Cassie and Wenzel types of wetting represent local energy minima for drops on rough surfaces. See, Patankar, N. A., On the modeling of hydrophobic contact angles on rough surfaces, Langmuir 19, 1249-1253 (2003). Drops gently deposited onto superhydrophobic rough surfaces resulted in extremely high contact angles which were well represented by the Cassie model, whereas drops that were allowed to fall onto the surface from a height gave lower contact angles that were better represented by the Wenzel model. See, He, B., Patankar, N. A. & Lee, J., Multiple equilibrium droplet shapes and design criterion for rough hydrophobic surfaces, Langmuir, 19, 4999-5003 (2003).

Contact angles on rough surfaces can transition from Cassie to Wenzel behavior when pressure is applied to the drop. See, Bico, J., Marzolin, C. & Quere, D., Pearl drops. Europhysics Letters 47, 220-226 (1999). It was found that when using smaller drops (˜5 μL), visible irradiation of the coated nanowire surface resulted in advancing contact angles of greater than 170°. Larger drops (˜15 μL) produced advancing contact angles of 157°. The weight of the larger drops can force the liquid into the depressions in the surface, making the Wenzel model applicable under these conditions.

(a) Cassie Model

Cassie's model is based on the assumption that the liquid does not fill the crevices of the rough surface, but rests on a composite surface composed of the solid material and air.

(b) Wenzel Model

In contrast, Wenzel's model hypothesizes that the liquid completely fills the depressions in the rough surface over the projected area of solid-liquid contact.

In the Wenzel model, the apparent contact angle on the fractal surface, θ_{f} may be expressed as,

$$cos {θ}_{f} = {( \frac{L}{l} )}^{D - 2} cos θ \qquad ( Eqn . 1 )$$

where θ is the contact angle on a flat surface with identical chemistry and D is the fractal dimension of the surface between the upper and lower scale limits, L and l, respectively. This indicates that if the flat surface contact angle is changed from a value θ₁ to θ₂ by the action of an external stimulus such as light, the apparent contact angle change on the fractal surface (θ_{f1}−θ_{f2}) may be expressed by

$$( cos {θ}_{f 1} - cos {θ}_{f 2} ) = {( \frac{L}{l} )}^{D - 2} ( cos {θ}_{1} - cos {θ}_{2} ) \qquad ( Eqn . 2 )$$

Since the term (L/l)^{D-2} is always >1 for a rough surface, this indicates that the use of a superhydrophobic rough surface will always amplify the magnitude of the stimulus-induced contact angle change relative to the smooth surface (until the theoretical limit of a 180° contact angle is reached). Thus, by combining photoswitchable surface chemistry with control of surface morphology, it is possible to amplify the photo-induced changes in the water contact angle.

Each of these theories leads to different values of θᵣ, and it has been experimentally demonstrated that both the Cassie angle, θᵣ^{C}, and the Wenzel angle, θᵣ^{W}, can be formed on the same surface depending on the means of formation of the droplet. See He, B., Patankar, N. A., and Lee, J., Multiple equilibrium droplet shapes and design criterion for rough hydrophobic surfaces, Langmuir, 2003. 19: p. 4999-5003. It has also been experimentally confirmed in different systems, that the Cassie and Wenzel contact angles each represent local energy minima separated by energy barriers corresponding to partial wetting of the roughness features. See Id.; Bico, J., Marzolin, C. and Quere, D., Pearl drops, Europhysics Letters, 1999. 47(2): p. 220-226.

The global energy minimum for this system was calculated to be the smaller of the two contact angles, θᵣ^{C} and θᵣ^{W} Experimental measurements on PDMS microstructures showed some quantitative agreement with this calculation. Differences between the experimental and calculated values were attributed to the 30° contact angle hysteresis present on even “flat” PDMS surfaces.

The interfacial energies of spiropyran-coated surfaces can be changed solely by altering the wavelength of irradiation. See Rosario, R. et al., Photon-modulated wettability changes on spiropyran-coated surfaces, Langmuir 18, 8062-8069 (2002). The maximum difference obtained between water contact angles under UV and Visible tight was of the order of 13°.

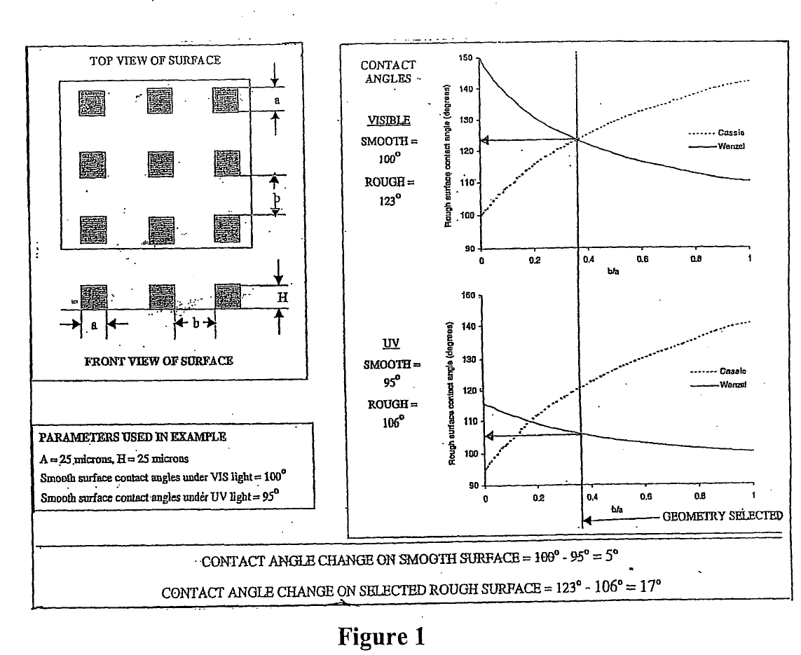

Selection of a particular surface roughness allows amplification of the magnitude of the light-induced contact angle changes on spiropyran-coated surfaces. The roughness characteristics axe defined by the geometry of surface features. FIG. 1 shows an example of how the selection of a particular rough surface can increase the light-induced contact angle change. In this example a 5° light-induced change is predicted to be amplified into a 17° change in contact angle on the rough surface.

(c) Determination of Optimal Surface Morphology

The apparent contact angle on non-porous Euclidian rough surfaces is given by the Wenzel equations. See Wenzel, R. N., Industrial and Engineering Chemistry Research, 1936, 28, 988.

cos θw=r cos θs (Eqn. 3)

The roughness coefficient r is defined as the ratio of the actual solid-liquid interfacial area to the projected solid-liquid interfacial area, and θ_{w} and θₛ are the solid-liquid contact angles on the rough surface and smooth surface, respectively. The effect of r is to enhance the inherent wetting behavior of the surface (by increasing the contact angle >90°, and decreasing the contact angle <90°).

However, for fractal surfaces, the term r is very large and can even be infinite for a mathematically ideal fractal surface. Additionally, if the fractal behavior extends to the molecular scale, fluids having different molecular dimensions would experience different solid-liquid interfacial areas. Thermodynamic models for the equilibrium contact angle, which take into account both the fractal nature of the surface and the relative dimensions of the different fluid molecules, have been developed. See, e.g., Hazlett, R. D., Journal of Colloid and Interface Science, 1990, 137, 527. The equilibrium contact angle is given by

$$cos {θ}_{fractal} = [ ( \frac{1 - Γ {f}^{1 - D / 2}}{1 - Γ} ) {( \frac{{σ}_{1}}{{σ}_{R}} )}^{1 - D / 2} ] cos θ s \qquad ( Eqn . 4 )$$

Here, f≡(σ₂/σ₁) and Γ≡(γₛ₂/γₛ₁). σ refers to the area of the interfacial tension, D is the fractal dimension, and the subscripts s, 1, and 2 refer to the surface, liquid, and vapor, respectively. σ_{R} is a reference area that represents the scale that would yield the Euclidean area if the fractal nature and dimension held to this scale, such that

πR² sin²θ=Cσ_{R}^{1-D/2} (Eqn. 5)

where R is the radius of the drop.

The first term within the correction factor in Eqn. 5 can either depress or elevate the contact angle depending on the relative sizes of the fluid molecules and their wetting tendencies. The second term is a measure of the extent of the fractal nature of the surface and is always greater than 1. When the lower limit of fractal behavior is larger than the areas of the fluid molecules, then the fluid molecules are able to probe all the irregularities on the surface and Eqn. 4 reduces to

$$cos {θ}_{W , fractal} = {( \frac{L}{l} )}^{D - 2} cos θ s \qquad ( Eqn . 6 )$$

where L and 1 are the upper and lower limits of fractal behavior. The correction term here is analogous to the roughness correction term of the Wenzel equation and quantifies the ratio of the actual solid surface area to the projected surface area. For example, and alkyl ketene dimmer fractal surface was found to possess a correction factor of (L/l)^{D-2}=(34/0.2)^{2.29-2≅4.43}, with the fractal limits being expressed in microns. See Onda, T., Shibuichi, S., Satoh, N. & Tsujii, K., Super-water-repellent fractal surfaces, Langmuir 12, 2125-2127 (1996).

In the case of rough porous surfaces, Cassie's equation describes the equilibrium contact angle on a composite surface, which retains pockets of air underneath the sessile drop. See Cassie, A. B. & Baxter, S., Wettability of porous surfaces, Transactions of the Faraday Society 40, 546-551 (1944).

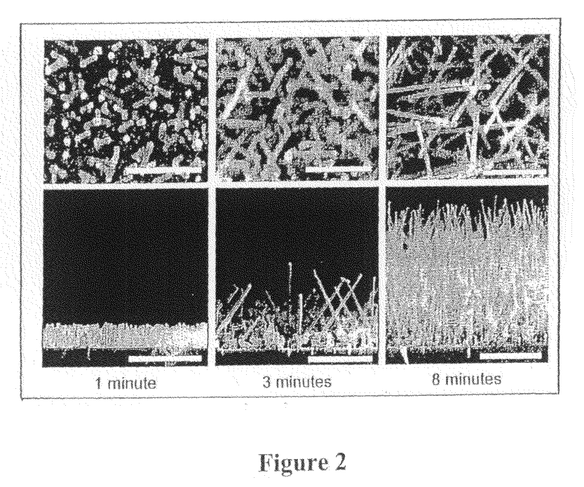

cos θc=f cos θ₁+f₂ (Eqn. 7)

where f₁ and f₂ are the ratios of projected areas of the solid surface-liquid and air surface-liquid interfaces, respectively, to the total projected area, θ_{c} is the Cassie contact angle and θ₁ is the solid-liquid contact angle. FIG. 2 shows a drop of liquid sitting on a fractally rough composite surface made up of solid and air. From the figure, f₁=a/(a+b)=f and f₂=b/(a+b)=(1−f). Substituting these ratios into Eqn. 7, the equation becomes

cos θ_{c}=f cos θ₁+f−1 (Eqn. 8)

Since the surface is fractally rough, in this case, θ₁ is equivalent to the Wenzel contact angle on fractally rough surfaces as given by Eqn. 6. Therefore, the Cassie equation can be extended to uniformly heterogeneous fractal surfaces by substituting Eqn. 6 into Eqn. 8.

$$cos {θ}_{C , fractal} = {f ( \frac{L}{l} )}^{D - 2} cos θ s + f - 1 \qquad ( Eqn . 9 )$$

This is the equivalent fractal form of Cassie's equation. While f can be calculated for well-defined Euclidean surfaces, fractal surfaces are not amenable to this quantitative treatment. Therefore, this may have to be an adjustable parameter.

Both Wenzel and Cassie equations represent local energy minima in drop conformation. For fractal surfaces, the Wenzel contact angle is always lesser or equal to the Cassie contact angle. The equilibrium drop shape with the lower value of apparent contact angle on rough Euclidean surfaces will have lower energy. See Patankar, N. A., On the modeling of hydrophobic contact angles on rough surfaces, Langmuir 19, 1249-1253 (2003). Extending this result to fractal surfaces, the Wenzel contact angle represents the global energy minimum of the system.

At intrinsic contact angles of >90°, the apparent contact angles (Wenzel and Cassie) increase as a function of the roughness of the surface as represented by the fractal dimension, D, until the physical limit of an apparent 180° contact angle is reached. The magnification of any light-induced contact angle change, as a function of D, has a maximum at the roughness that first produces an apparent 180° contact angle on the more hydrophobic surface. Therefore, the degree of fractal surface roughness that produces the maximum magnification of light-induced contact angle changes, Dₒₚₜᵢₘₐₗ, can be predicted by

$${D}_{optimal} = \frac{ln ( - 1 / cos {θ}_{S} )}{ln ( L / l )} + 2 = \frac{ln [ sec ( {θ}_{S} + π ) ]}{ln [ L / l ]} + 2 \qquad ( Eqn . 10 )$$

(d) Fractally Rough Surfaces

Cross sectional images of fractally rough oxidized silicon nanowire-bearing surfaces were obtained using SEM (FIG. 2). A box counting fractal analysis was performed on trace curves of the cross sectional SEM images, and the cross sectional fractal dimension of the surface, D_{cross}, was determined to be 1.54 between the lower and upper limits of fractal behavior of 74 nm and 202 nm, respectively. The three-dimensional fractal dimension of the surface was estimated to be D˜D_{cross}+1=2.54. See, Vicsec, T., Fractal growth phenomena (World Scientific, Singapore, 1989).

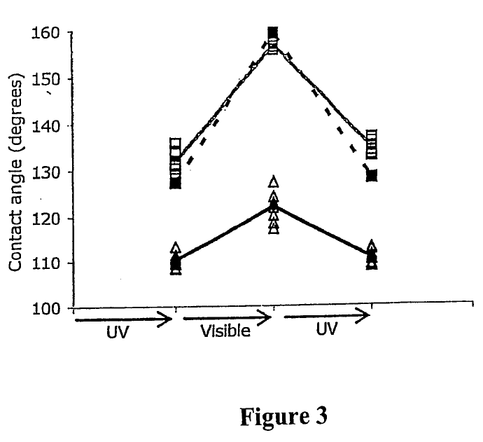

The use of these fractal roughness parameters in the Wenzel model for contact angles on fractal surfaces (Eqn. 1) gave an excellent fit with the experimental contact angles on the rough surface as shown by the dashed line in FIG. 3. This demonstrates that the large water drops filled the crevices in the nanowire structure, as described by the Wenzel model.

2. Theory and Calculations

a) Wenzel Wetting

The Wenzel treatment was derived for a system in which the liquid completely fills the depressions in the rough surface over the area of solid-liquid contact. Again, the apparent contact angle on non-porous Euclidian rough surfaces is given by equation 10. See Wenzel, R. N. Ind. Eng. Chem. Res. 1936, 28, 988-994.

cos θ_{W}=r cos θ_{S} (Eqn. 3)

The roughness coefficient r is defined as the ratio of the actual solid-liquid interfacial area to the solid-liquid interfacial area projected onto the surface plane, and θ_{W} and θ_{S} are the solid-liquid contact angles on the rough and smooth surface, respectively. The effect of r is to enhance the inherent range of wetting behavior of the surface by increasing contact angles initially >90° and decreasing contact angles initially <90°. Accurate prediction of Wenzel or Cassie-Baxter contact angles using r values for a variety of regularly patterned surfaces has been demonstrated. See Patankar, N. A. Langmuir 2004, 20, 8209-8213; Marmur, A. Langmuir 2004, 20, 3517-3519; Shirtcliffe, N. J.; McHale, G.; Newton, M. I.; Perry, C. C. Langmuir 2005, 21, 937-943.

However, for fractal surfaces the term r is often larger than that for regularly patterned surfaces. Additionally, if the fractal behavior extends to the molecular scale, fluids having different molecular dimensions can experience different solid-fluid interfacial areas. A thermodynamic model for the equilibrium contact angle on rough surfaces that takes into account both the fractal nature of the surface and the relative dimensions of the different fluid molecules has been developed (eqn. 11). See Hazlett, R. D. J. Colloid Interface Sci. 1990, 137, 527-533.

$$cos {θ}_{W , fractal} = [ ( \frac{1 - Γ {f}^{1 - D / 2}}{1 - Γ} ) {( \frac{{σ}_{1}}{{σ}_{R}} )}^{1 - D / 2} ] cos {θ}_{S} \qquad ( Eqn . 11 )$$

Here f=(σ₁/σ₂), Γ≡(γₛ₂/γₛ₁), σ refers to area of the molecular species, γ is the interfacial tension, D is the fractal dimension, θ_{Wfractal} is the solid-liquid contact angle on the fractally rough surface, and the subscripts 1 and 2 refer to the liquid and vapor phases, respectively. σ_{R} is a reference area that represents the scale at which the fractal surface area would equal the Euclidean surface area, assuming the fractal nature and dimension held to this scale.

The term within the first parentheses in eqn. 11 can either depress or elevate the rough surface contact angle depending on the relative sizes of the fluid molecules and their wetting tendencies. The second term within the correction factor is a measure of the extent of the fractal nature of the surface, and is greater than 1. If the lower limit of fractal behavior is larger than the areas of the fluid molecules, then the fluid molecules would be able to probe all the irregularities on the fractal surface and the contact angle has been shown to be represented by eqn. 12, where L and l are the upper and lower limits of fractal behavior. See Onda, T.; Shibuichi, S.; Satoh, N.; Tsujii, K. Langmuir 1996, 12, 2125-2127.

$$cos {θ}_{W , fractal} = {( \frac{L}{l} )}^{D - 2} cos θ s \qquad ( Eqn . 12 )$$

The roughness term here, (L/l)^{D-2}, is analogous to the r roughness term of the Wenzel equation (Eqn. 3), and quantifies the ratio of the actual solid surface area to the projected surface area.

b) Cassie-Baxter Wetting.

In the case of rough porous surfaces, the Cassie-Baxter equation describes the equilibrium contact angle on a composite surface which retains pockets of air underneath the sessile drop,

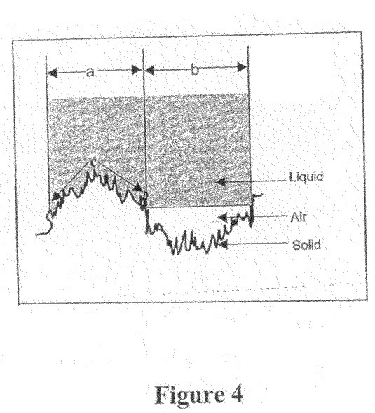

cos θ_{c}=f₁ cos θ₁−f₂ (Eqn. 13)

where f₁ and f₂ are the ratios of the projected areas of the solid surface-liquid and air surface-liquid interfaces, respectively, to the total projected area, θ_{C} is the Cassie contact angle and θ₁ is the solid-liquid contact angle. See Cassie, A.; Baxter, S. Trans. Faraday Soc. 1944, 40, 546-551. FIG. 4 shows schematically a drop of liquid sitting on a fractally rough composite surface made up of solid and air. From this figure, f₁=a/(a+b)=f and f₂=b/(a+b)=(1−f). Substituting these ratios into eq. 13 we arrive at

cos θ_{c}=f cos θ₁+f−1 (Eqn. 14)

Since the surface is fractally rough, θ₁ is equivalent to the Wenzel contact angle on fractally rough surfaces as given by eq. 12. Therefore, the Cassie-Baxter equation can be extended to uniformly heterogeneous fractal surfaces by substituting eq. 12 into eq. 14

$$cos {θ}_{C , fractal} = {f ( \frac{L}{l} )}^{D - 2} cos θ s + f - 1 \qquad ( Eqn . 15 )$$

This is the equivalent fractal form of the Cassie-Baxter equation. While it is possible to calculated for well-defined Euclidian surfaces, fractal surfaces are not amenable to this treatment.

c) Hysteresis in Wenzel Wetting.

The difference between the advancing and receding angles can be used to gauge how readily a liquid drop would move under an applied stimulus. See Ichimura, K.; Oh, S.; Nakagawa, M. Science 2000, 288, 1624-1626; Oh, S.-K.; Nakagawa, M.; Ichimura, K. J. Mater. Chem. 2002, 12, 2262-2269. A practical, phenomenological method for describing hysteresis was developed by Kamusewitz et al., see Kamusewitz, H.; Possart, W. App. Phys. A—Mat. Sci. Proc. 2003, 76, 899-902, extending the work of Adam and Jessop, see Adam, N. K.; Jessop, G. J. Chem. Soc. 1925, 127, 1863-1868, and Good, see Good, R. J. J. Am. Chem. Soc. 1952, 74, 5041-5042, by relating the equilibrium smooth surface contact angle (θ_{S,e}) directly to the Wenzel receding (θ_{W,r}) and Wenzel advancing (θ_{W,a}) contact angles on rough surfaces using the following equation

cos(θ_{W,a})+cos(θ^{W,r})=2r cos(θ_{S,e}) (Eqn. 16)

The equilibrium contact angle for a particular surface chemistry can be found by varying the roughness and determining at what angle the linear plots of the advancing angle versus hysteresis (Δθ) and receding angle versus hysteresis intersect. Tadmor derived the equilibrium contact angle based on a line energy analysis. See Tadmore, R. Langmuir 2004, 20, 7659-7664. This theory agrees reasonable well with the assumption that the cosine of the equilibrium angle is equal to the average of the cosines of the smooth contact angles for the advancing and receding cases.

The advancing and receding contact angles after varying surface chemistry and roughness were measured. Fractally rough surfaces a provide a reasonable fit of the Wenzel wetting data by assuming that the equilibrium contact angle is quite similar to the smooth advancing contact angle θ_{S,a},

cos(θ_{W,a})+cos(θ_{W,r})=2r cos(0.97θ_{S,a}) (Eqn. 17)

Because fractally rough surfaces have a dramatically lower hysteresis at high surface areas the advancing angle is very nearly equal to the equilibrium angle. This type of behavior has been observed for a fractal surface created by electrodeposited copper surfaces with a hydrophobic coating, in sharp contrast to the behavior they observed with regularly patterned copper surfaces. See Shirtcliffe, N. J.; McHale, G.; Newton, M. I.; Perry, C. C. Langmuir 2005, 21, 937-943.

3. Hydrophobic Layer

The hydrophobic layer can be anything that creates a lack of affinity for water. For example, a mixture of perfluorooctyltrchloroisilane and a tert butyl diphenylchlorosilane can be attached to the surface, such as a silicon surface. Other halosilanes can be used for example, to create a hydrophobic layer on the surface. For example, any molecule which has a reactive portion which is anything capable of binding or covalently attaching to the surface and a hydrophobic portion which is, for example, a hydrocarbon or a perhalogenated hydrocarbon. Any means for making a surface hydrophobic consistent with the devices, compositions, and methods disclosed herein can be used and is considered disclosed.

A hydrophilic surface can be produced by using molecules lacking a hydrophobic portion, but instead have, for example, a hydrophilic portion, such as polar and ionic groups.

4. Amplification Layer

The devices also can contain an amplification molecule which is any molecule which can amplify the hydrophobicity of the hydrophobic layer, typically in an inducible way. For example, isomerizable molecules, stimulus inducible molecules, photosensitive molecules are examples of amplifying molecules. A stimulus inducible molecule is any molecule which under goes a change, as disclosed herein, upon a receiving a stimulus.

For example, disclosed are devices comprising a surface, wherein the surface has roughness, a hydrophobic layer, and an isomerization molecule which can be isomerized into a first and a second form, wherein the first and second forms have different effects on the wetting of the surface by a fluid. For example, disclosed are devices, wherein the isomerization molecule is an organic molecule. Also disclosed are devices wherein the isomerization molecule is covalently attached. Also disclosed are devices wherein the isomerization molecule is a photoresponsive molecule, and devices wherein the photoresponsive molecule is a photochrome. Also disclosed are devices wherein the photochrome isomerizes under two different wavelengths of light.

Disclosed are devices wherein the stimulus inducible molecule, such as the isomerization molecule, such as a photochrome, such as an organic photochrome, comprise a spiropyran, and, for example, wherein the spiropyran is an indolinospiropyran.

Disclosed are devices wherein the stimulus inducible molecule, such as the isomerization molecule, such as a photochrome, such as an organic photochrome comprises or is a spirooxazine, benzo-naphthopyran, naphthopyran, azobenzene, fulgide, diarylethene, dihydroindolizine, photochromic quinone, perimidinespirocyclohexadienone, or dihydropyrene or combinations thereof.

Disclosed are devices wherein the stimulus inducible molecule, such as the isomerization molecule, such as a photochrome, such as an organic photochrome, comprises a heteroaryl group.

It is understood that any of the molecules disclosed herein, such as the spiropyrans with any basic spiropyran molecular skeleton and any pattern of substituents can be used, as long as the molecule can be attached, such as covalently to the surface, such as the rough surface as disclosed herein. It is understood that the molecules can be covalently attached through linkers, or for example, can be attached with capture-tag systems, such as a streptavidin-biotin system or some other non-covalently system, wherein the surface has been derivatized and the stimulus induced molecule has been derivatized such that they can interact through a capture-tag interaction.

Disclosed are devices comprising a fractally rough, hydrophobic surface, and a liquid droplet, wherein the liquid droplet has a contact angle with the surface, and wherein the contact angle is magnified relative to a smooth surface and wherein the contact angle hysteresis is lowered relative to a smooth surface.

Also disclosed are devices, wherein the hydrophobic surface is a superhydrophobic surface.

It is understood that as used herein, a superhydrophobic surface is a surface that will produce a contact angle of at least 160 degrees. It is also understood, however, that hydrophobic surfaces that produces a contact angle with water of at least 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, and 180 are also disclosed.

Disclosed are devices wherein the difference between the contact angle and the contact angle hysteresis is at least 1.2, 1.4, 1.6, 1.8, or 2.

Also disclosed are devices comprising a fractally rough, hydrophobic surface, and a liquid droplet, wherein the liquid droplet has a contact angle with the surface, and wherein the advancing contact angle under a first condition is lower than the receding contact angle under a second condition.

Disclosed are devices, for example, wherein the first and second conditions are light related.

Also disclosed are devices, for example, wherein the first condition is an ultra violet light condition and wherein the second condition is a visible light condition. Also disclosed are devices, wherein the first condition is a visible light condition and wherein the second condition is an ultra violet light condition.

Disclosed are devices, wherein the difference in the contact angle hysteresis as defined by the advancing contact angle and the receding contact angle is less than the difference in the contact angle determined by two different light conditions. Also disclosed are devices, wherein, for example, the contact angle hysteresis is less than 37 degrees or less than 17 degrees.

Disclosed are devices comprising a surface, wherein the surface has roughness, a hydrophobic layer, and a photoswitchable surface chemistry. A photoswitchable surface chemistry requires a surface that includes photoresponsive molecules such as photochromes. These molecules can be isomerized (structurally switched) between two different molecular structures using light. One wavelength range of light converts the molecules on the surface predominantly into one isomeric molecular form, and a second wavelength range of light converts this resulting form back into the initial molecular form. As disclosed herein, the two isomeric forms of the photoresponsive molecule interact differently with fluids on the surface, and thus change the surface wetting properties for that fluid. For example, one form can make the surface more wettable by water (hydrophilic), and the other form would then make the surface less wettable by water (hydrophobic).

Disclosed are devices comprising a surface, wherein the surface has roughness, a hydrophobic layer, and a stimulus inducible molecule, wherein the stimulus inducible molecule causes a contact angle change when stimulated, producing a stimulus induced contact angle change having a magnitude.

Disclosed are devices wherein the surface is a semiconductor, an oxide, a glass, a metal, an insulator, a refractory material, carbon, or diamond like carbon.

Also disclosed are devices, wherein the stimulus is light, heat, pH, a biologically active molecule, or solution chemistry.

Disclosed are devices comprising a surface, wherein the surface comprises a rough, hydrophobic, and a hydrophobicity variable molecule on a contact surface.

Disclosed are devices, wherein the device further comprises hydrophobic and hydrophilic regions that have variable fluid wetting angles.

Also disclosed are devices, wherein a fluid moves across the surface when the contact angle of the fluid at the advancing edge is below the contact angle of the receding edge.

For example, disclosed are devices, wherein the fluid is saliva, water, sea water, blood, semen, plasma, urine, or lymph. Any fluid, such as an aqueous fluid, can be used with the disclosed devices. For example, fluids having contact angles on a given surface, which are at least 50%, 55%, 60%, 65%, 70%, 75%, 80%, 85%, 90%, 91%, 92%, 93%, 94%, 95%, 96%, 97%, 98%, 99%, 100%, 101,%, 102%, 103%, 104%, 105%, 106%, 107%, 108%, 109%, 110%, 115%, 120%, 125%, 130%, 135%, 140%, 145%, or 150% of the contact angles of pure water on the surface.

Also disclosed are devices wherein the contact angle switching is amplified, and/or wherein the contact angle hysteresis is decreased, and/or, wherein the contact angle switching is amplified and wherein the contact angle hysteresis is decreased, and/or wherein a liquid drop moves based only on the influence of light.

Also disclosed are devices, wherein the device comprises a surface tension driven microfluidic system. For example, disclosed are devices, wherein the microfluidic system comprises a light directed processing of fluid droplets on the surface, wherein the fluid is either mixed, reacted, or analyzed.

Disclosed are compositions comprising a variable hydrophobicity agent coated on a rough surface.

Also disclosed are compositions, wherein the variable hydrophobicity agent is photosensitive.

Also disclosed are compositions, wherein the surface comprises at least one feature.

Also disclosed are compositions, wherein the feature has an average feature height, an average feature width, and an average inter-feature distance.

Also disclosed are compositions, wherein the average feature height is greater than the average feature width.

Also disclosed are compositions, wherein the average feature height is about 1.1, 2, 3, 4, 5, 6, 7, 8, 9, 10, 12, 13, 14, or 15 times the average feature width.

Also disclosed are compositions, wherein the average inter-feature distance to average feature width ratio is from about 0.1 to about 100, 0.1 to 50, 5-50, 10-30, or 0.2 to 0.5.

Also disclosed are compositions, wherein the average inter-feature distance to average feature width ratio is about 0.2 to 4, 0.3 to 3, 0.3 to 2, or about 0.36.

Also disclosed are compositions, wherein the variable hydrophobicity agent comprises an organic photochrome having a polar form and a nonpolar form.

Also disclosed are compositions, wherein the variable hydrophobicity agent has predominantly the polar form when exposed to light having a first wavelength.

Also disclosed are compositions, wherein the variable hydrophobicity agent has predominantly the nonpolar form when exposed to light having a second wavelength.

Also disclosed are compositions, wherein the first wavelength is less than the second wavelength.

Also disclosed are compositions, wherein the variable hydrophobicity agent comprises a spiropyran compound.

Also disclosed are compositions, further comprising a fluid droplet in contact with the surface.

Also disclosed are compositions, wherein the fluid droplet contact with the surface has a contact angle of at least about 155 to 170 degrees.

Also disclosed are compositions, wherein the fluid droplet contact with the surface has a contact angle of about 170 to 180 degrees.

Disclosed are compositions comprising a photosensitive variable hydrophobicity agent coated on a rough surface, wherein the surface has a three dimensional fractal dimension of at least about 2.2.

Also disclosed are compositions, wherein the surface has a three-dimensional fractal dimension of about 2.54.

Also disclosed are hydrophobicity variable molecules which are molecules which under different conditions have different effects on the hydrophobicity characteristics of a surface to which they are attached. A spiropyran is an example of a a hydrophobicity variable molecule.

(1) Photoresponsive Molecules

(a) Organic Photochromes

(i) Spiropyras

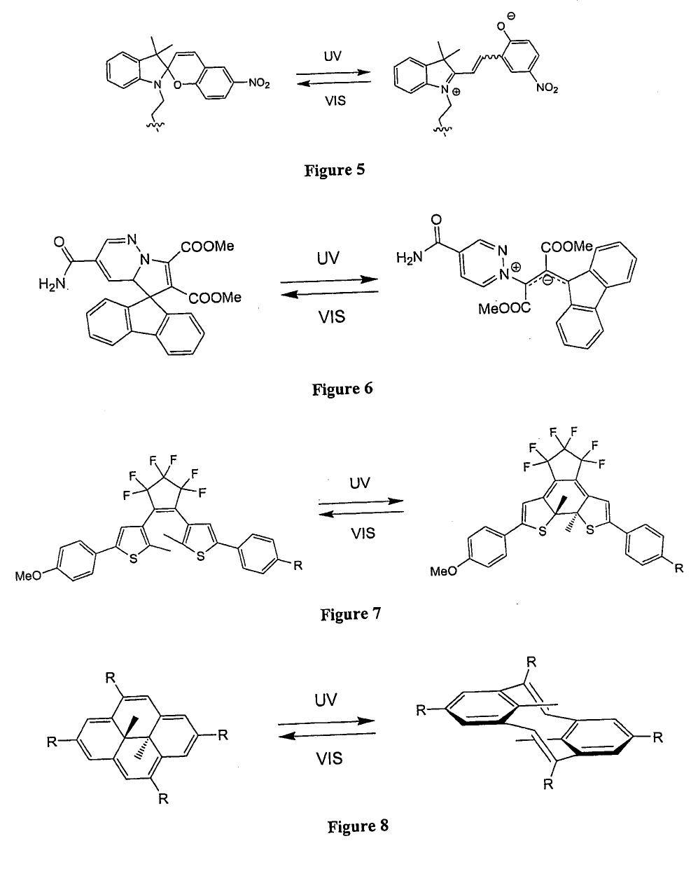

Spiropyrans are a class of organic photochromes that undergo a reversible transition from a closed, nonpolar form to a highly polar, open form when irradiated with higher energy, shorter wavelength light (e.g., ultraviolet (UV) light (e.g., 366 nm)). (FIG. 5).

Irradiation with lower energy, longer wavelength light (e.g., visible (VIS) light (e.g., 450-550 nm)) converts the molecule back to its closed, nonpolar form. Visible light irradiation of the spiropyran coating yields a relatively hydrophobic surface (higher contact angle) that can be reversibly converted into a more hydrophilic surface (lower contact angle) with UV light irradiation. The reversible switching of contact angles using UV and visible light for these molecular monolayers on smooth glass surfaces is due to the photon-modulated conversion of the spiropyran molecules between open and closed forms. See, Rosario, R. et al., Photon-modulated wettability changes on spiropyran-coated surfaces, Langmuir 18, 8062-8069 (2002).

(ii) Dihydroindolizines

Dihydroindolizines are a class of organic photochromes that undergo a reversible transition from a closed, nonpolar, form to a highly polar, open form when irradiated with higher energy, shorter wavelength light (e.g., ultraviolet (UV) light). (FIG. 6).

(iii) Dithiensylethene

Dithienylethenes are a class of organic photochromes that undergo a reversible transition from an open, nonplanar form to a closed, planar form when irradiated with higher energy, shorter wavelength light (e.g., ultraviolet (UV) light). (FIG. 7).

(iv) Dihydropyrene

Dihydropyrenes are a class of organic photochromes that undergo a reversible transition from a closed, planar form to an open, nonplanar form when irradiated with higher energy, shorter wavelength light (e.g., ultraviolet (UV) light). (FIG. 8).

(b) Linkers

A “linker” can be any compound that has at least one group that can form a covalent bond with the photoresponsive molecule and at least one group that can form a covalent bond with the surface. In one aspect, the linker group can have up to 25 carbon atoms. In one aspect, the linker can be a hydrocarbon having two or more groups capable of reacting with the photoresponsive molecule and the surface. Examples of groups capable of reacting with the photoresponsive molecule include, but are not limited to, hydroxyl groups, amino groups, and carboxyl groups. For example, the hydrocarbon can have at least one hydroxyl group, amino group, carboxyl group, or combinations thereof.

A group capable of reacting with the surface can be a group capable of forming a covalent, ionic, or coordination bond with the surface. The group can include, for example, a silane group or a sulfur group. Examples of groups capable of reacting with the surface include, but are not limited to, amino, halosilane, silane, thio, halothio, or derivatives thereof. For example, the hydrocarbon can have at least one amino, halosilane, silane, thio, halothio, or combinations thereof. It is contemplated that the linker can be of varying molecular weight.

Examples of linkers include, but are not limited to, 2-aminopropanoic acid, 3-aminobutanoic acid, and the like. It is contemplated that the linker molecule can be covalently attached to the photoresponsive molecule or the surface prior to linking the photoresponsive molecule to the surface.

### D. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular photosensitive molecule is disclosed and discussed and a number of modifications that can be made to a number of molecules including the photosensitive molecule are discussed, specifically contemplated is each and every combination and permutation of photosensitive molecule and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

2. Compositions with Similar Functions

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

### E. Methods of Making the Compositions

The compositions disclosed herein and the compositions necessary to perform the disclosed methods can be made using any method known to those of skill in the art for that particular reagent or compound unless otherwise specifically noted.

1. Process Claims for Making the Compositions and Devices

Disclosed are processes for making the compositions and devices as well as making the intermediates leading to the compositions. There are a variety of methods that can be used for making these compositions, such as synthetic chemical methods and standard molecular biology methods. It is understood that the methods of making these and the other disclosed compositions are specifically disclosed.

### F. Methods of Using the Compositions

Disclosed are methods of inducing linear movement of a fluid droplet on a surface comprising the steps of: a) providing a rough surface coated with a photosensitive variable hydrophobicity agent; b) positioning a fluid droplet in contact with the surface; and c) exposing the surface and droplet to a variable wavelength light gradient.

Disclosed are methods for patterning the regions of switchable hydrophobicity or stimulus induced regions on the surface. For example, a channel can be formed by making a narrow trough (relative to the droplet diameter) with a region that is hydrophilic which can then be surrounded by a stimulus inducible molecule, such as a hydrophobic switching molecule which allows for simultaneous movement of a droplet along a confined and predetermined path.

Disclosed are methods and devices that can pin or hold or confine the droplets in ways in addition to the movement generated on the disclosed surfaces. These methods and devices are advantageous for analysis or synthesis of materials or chemicals.

Also disclosed are methods of using any of the devices or compositions disclosed herein, in any type of analysis or diagnostic application. For example disclosed are methods, wherein providing a surface having a three dimensional fractal dimension of at least about 2.2 comprises the steps of: a) providing a polished silicon surface; and b) increasing the roughness of the surface by vapor deposition of silicon. Also disclosed are methods, wherein vapor deposition of silicon results in nanowires having diameters of from about 20 nm to about 50 nm. Also disclosed are methods, wherein coating the surface with a photosensitive variable hydrophobicity agent comprises the steps of a) treating the surface with tert-butyldiphenylchlorosilane; b) treating the surface with perfluorooctyltrichlorosilane; c) treating the surface with 3-aminopropyldiethoxymethylsilane; and d) treating the surface with a photosensitive variable hydrophobicity agent.

Also disclosed are methods of preparing a surface comprising the steps of: a) providing a surface having a three dimensional fractal dimension of at least about 2.2; and b) coating the surface with a photosensitive variable hydrophobicity agent.

1. Methods of Using the Compositions as Research Tools

The disclosed compositions can be used in a variety of ways as research tools.

The compositions can be used for example as targets in combinatorial chemistry protocols or other screening protocols to isolate molecules that possess desired functional properties.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### G. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### a) Movement of Liquid by Light Induced Changes

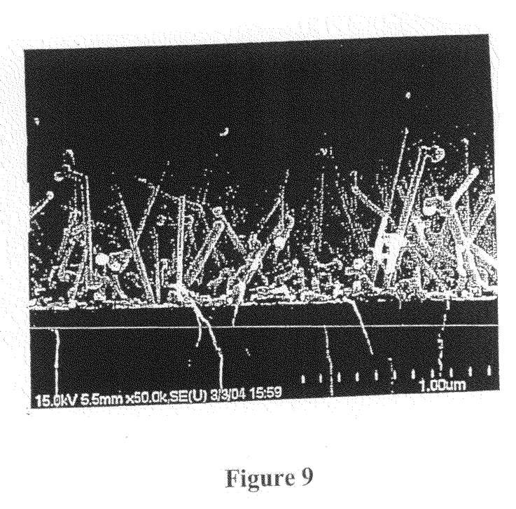

A polished silicon wafer bearing random silicon nanowires with diameters of 20-50 nm was prepared by a vapor-liquid-solid technique. See, e.g., Wagner, R. S. in Whisker Technology (ed. Levit, A. P.) 47-119 (Wiley-Interscience, New York, 1970). (FIG. 9) The air-oxidized silicon surface was treated with tert-butyldiphenylchlorosilane and perfluorooctyltrichlorosilane, followed by 3-aminopropyldiethoxymethylsilane, to which a photochromic spiropyran molecule was later attached by a published technique. See, Rosario, R. et al., Photon-modulated wettability changes on spiropyran-coated surfaces, Langmuir 18, 8062-8069 (2002).

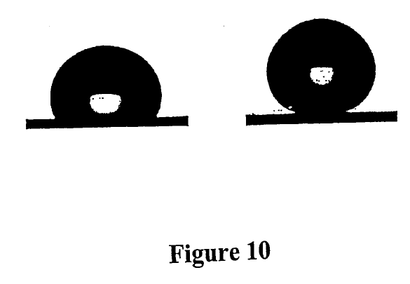

After surface derivatization with spiropyran-containing monolayers on silicon (Si) nanowire and adjacent smooth silicon surfaces, multiple measurements of advancing and receding water contact angles under UV and visible irradiation were performed using the sessile drop method. Direct comparisons of water contact angles on adjacent polished and nanowire areas are shown in FIG. 10. The combination of the surface roughness and the hydrophobic coating resulted in significantly higher contact angles on the nanowire surface compared to the smooth surface.

The average advancing contact angle on the smooth surface was 12° lower under UV irradiation than under visible irradiation (FIG. 3). On the nanowire-bearing surface, this light-induced contact angle change increased to 23° (FIG. 3). The increase in the light-induced contact angle changes on the nanowire-bearing surface confirmed that roughness has the effect of amplifying stimulus-induced contact angle changes relative to smooth surfaces by nearly a factor of two.

Under visible irradiation of the spiropyran-coated surfaces, the water contact angle hysteresis was measured to be 37° on the smooth surface, whereas on the nanowire-bearing surface a significantly lower value of only 17° was observed.

### b) Control

In a control experiment on the smooth spiropyran-coated surface, the advancing water contact angle under UV irradiation (110°) was higher than the receding water contact angle under visible irradiation (85°). This does not fulfill the criterion for liquid motion, and it was found that water drops on the smooth surface could not be moved using light.

In contrast, on the spiropyran-coated nanowire-bearing surface, the advancing water contact angle under UV irradiation (133°) was lower than the receding water contact angle under visible irradiation (140°). Accordingly, when an ultraviolet light-visible light gradient was applied across water drops sitting on the nanowire-bearing surface, the drops moved towards the UV end of the gradient.

Control experiments performed on drops sitting on nanowire-bearing surfaces coated with the hydrophobic layer, but without the spiropyran, did not result in any drop motion. Therefore, it can be concluded that the motion of the water droplets on the photoresponsive, nanowire-bearing surface was due to the roughness-magnified light-induced switching of surface energy by the spiropyrans coupled with the lower contact angle hysteresis of the superhydrophobic surface.

### c) Conclusions

Thus, it is demonstrated that surface roughness can be an effective tool for the amplification of stimulus-induced contact angle switching. The degree of amplification due to roughness was predicted using a Wenzel model. The combination of roughness-amplification of contact angle change with the reduced contact angle hysteresis of the nanowire-bearing, photoresponsive surfaces resulted in advancing contact angles under UV irradiation that were lower than the receding angles under visible irradiation. This for the first time permitted water drops on the nanowire surface to be moved solely using gradients of UV and visible light.

This result can lead to the development of photonic control of water movement in microfluidic devices. Additionally, since the fluid driving force in electrowetting, (see, e.g., Lahann, J. et al., A reversibly switching surface, Science 299, 371-374 (2003); Schneemilch, M., Welters, W. J., Hayes, R. A. & Ralston, J., Electrically induced changes in dynamic wettability. Langmuir 16, 2924-2927 (2000)) and thermowetting (see, e.g., Yakushiji, T. & Sakai, K., Graft architectural effects on thermoresponsive wettability changes of poly(N-isopropylacrylamide)-modified surfaces, Langmuir 14, 4657-4662 (1998); Liang, L., Ski, M., Viswanathan, V. V., Peummg, L. M. & Young, J. S., Temperature-sensitive polypropylene membranes prepared by plasma polymerization, Journal of Membrane Science 177, 97-108 (2000)) microfluidic systems is also the stimulus-induced difference between advancing and receding contact angles, these findings can enhance fluidic motion and control in these systems.

### Example 2

### a) Hydrophobic/Spiropyran Coating Procedure

Both nanowire and flat silicon oxide samples were cleaned using a 1:1 volume ratio of methanol/concentrated hydrochloric acid solution, followed by extensive washing in deionized water, yielding a nanowire surface contact angle of about 0° and a flat surface contact angle of 19°. The samples were then treated with a toluene solution of tert-butyldiphenylchlorosilane and perfluorooctyltrichlorosilane in the ratio of 10:1, giving a nanowire surface contact angle of about 175° (i.e., Cassie-Baxter) and a flat surface contact angle of 106±2°. This was followed by treatment with a toluene solution of (3-aminopropyl)diethoxymethylsilane and curing at 140° C., yielding a nanowire surface contact angle of about 175° and a flat surface contact angle of 103±3°. The silane-treated nanowire and flat silicon oxide samples were then incubated in an ethanolic solution of a photochromic spiropyran acid (1 mM) in the presence of the coupling agent 1-ethyl-3-(3-(dimethylamino)propyl)carbodiimide (10 mM), washed sequentially with ethanol and water, and dried under vacuum, producing a nanowire surface contact angle of about 174° and a flat surface contact angle of 107±8° under visible irradiation.

### b) Growth of Nanowires

Silicon nanowires were prepared by a vapor-liquid-solid (VLS) growth technique, using small dots of gold that act as catalytic seeds for growing a high density of nanowires on silicon substrates (FIG. 2). During evaporation of a few monolayers of Au on a clean Si or glass surface, the Au self assembles into nanodots. In the subsequent VLS synthesis the Au dots form a eutectic liquid with Si from which liquid-mediated growth of single crystal Si nanowires occurs. The nanowire diameters are set by the Au dot diameters, with one-dimensional growth occurring as the AuSi eutectic dot rides along at the free end of the growing wire. The growth rate is linear in time and the length of the nanowires is thus easily controlled by fixing the growth time. The Au dots at the end of the nanowires account for only a very small area. Typical VLS silicon nanowire growth conditions for these studies were 400 to 500° C. with disilane gas pressures of 3 mTorr, resulting in nanowire diameters of 20-50 nm and lengths of 1-3 μm.

### c) UV-Ozone Treatment

In one approach to the study of the effect of surface chemistry changes on nanowire surfaces without altering the surface geometry, a UV-ozone cleaner (Jelight Company Inc., model 42) was used. This apparatus contains a UV source and a chamber with adjustable oxygen flow and pressure. Atomic oxygen is generated when molecular oxygen and ozone are dissociated by UV light. Any organic coating on the nanowires reacts with atomic oxygen, forming volatile molecules that desorb from the surface. The process is known not to damage delicate structures in semiconductor processing. A nanowire coating can thus be removed to different degrees, leading to a continuous variation in hydrophobicity, by varying the treatment time while conducting the cleaning at room temperature.

### d) Contact Angle Measurements

Advancing and receding contact angle measurements were performed using a Rame-Hart Model 250 standard automated goniometer. For measuring the advancing angle on flat surfaces, 5 microliters of deionized water was dropped onto the sample from a microsyringe bearing a needle with a hydrophobic tip. For superhydrophobic surfaces, a larger drop of about 15-20 microliters was used because smaller drops easily rolled off the surface. This led to a small degree of measurement error since the drop was not fully spherical. An image of the drop was taken shortly after the drop was deposited in order to avoid measurement error due to drying. For receding angles, the microsyringe needle was used to draw some of the water out of the drop. The software automatically generates tangent measurements on the drop profiles. Usually four measurements were taken on different parts of the sample surface in order to characterize the overall properties of the surface.

### e) Results and Discussion

In order to investigate how wetting behavior is influenced by the surface properties of roughness and hydrophobicity, samples were prepared using the same chemical modification procedure on both smooth and nanowire surfaces. For surface modification, fluorocarbon and hydrocarbon-bearing silanes, with or without addition of the photochromic spiropyran molecule, were employed. This allowed investigation of the superhydrophobic surface regime and use of light-initiated surface chemistry alteration as a method for changing some aspects of surface chemistry without changing the overall nanomorphology or distribution of the molecules on the surface.

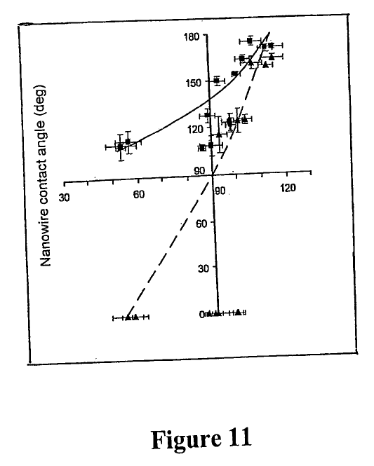

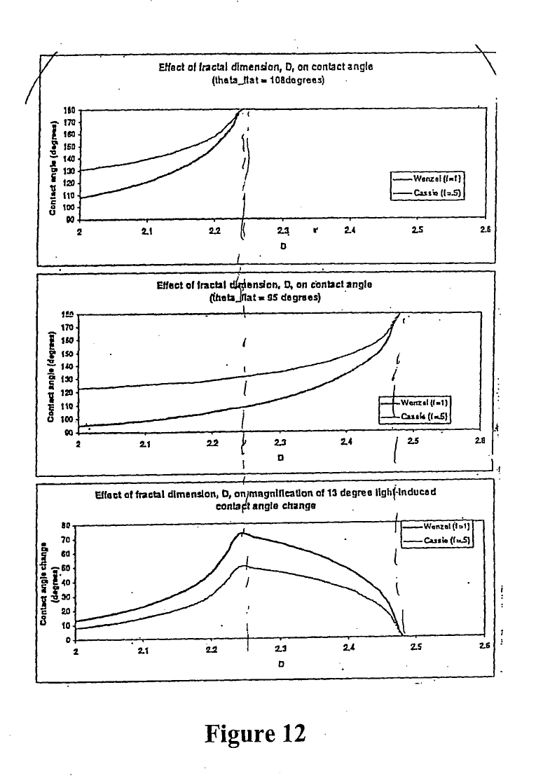

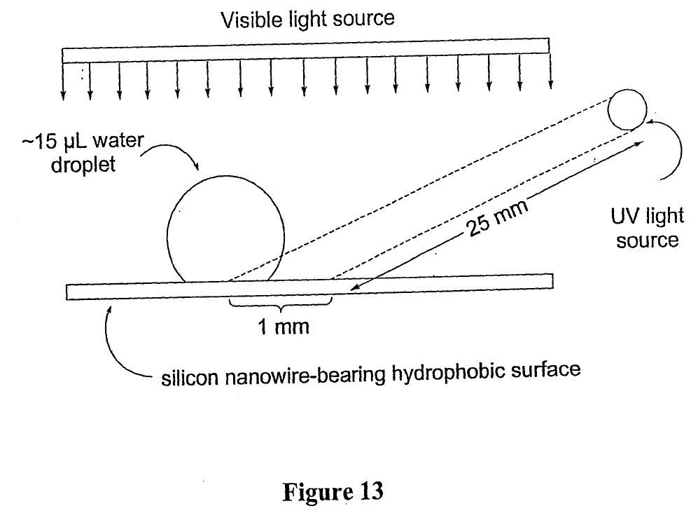

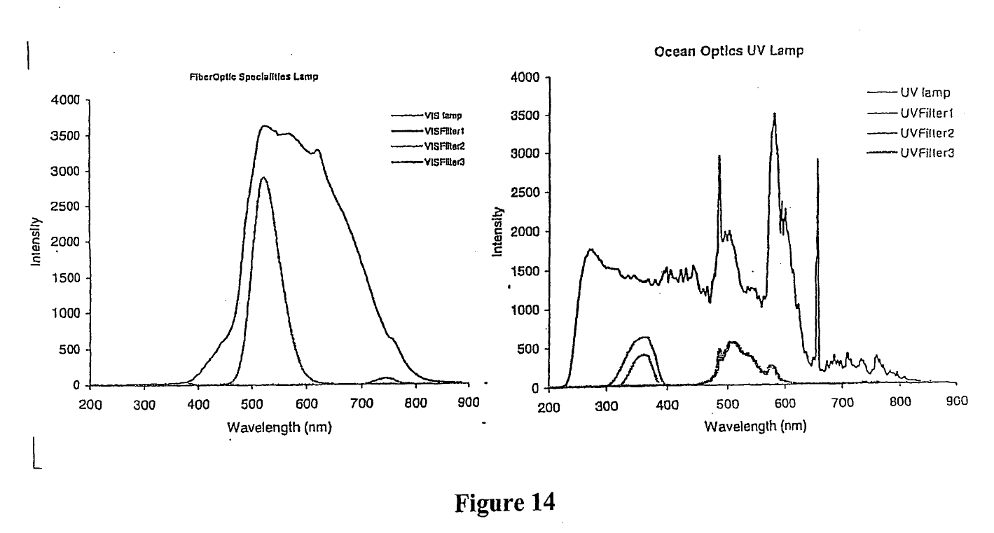

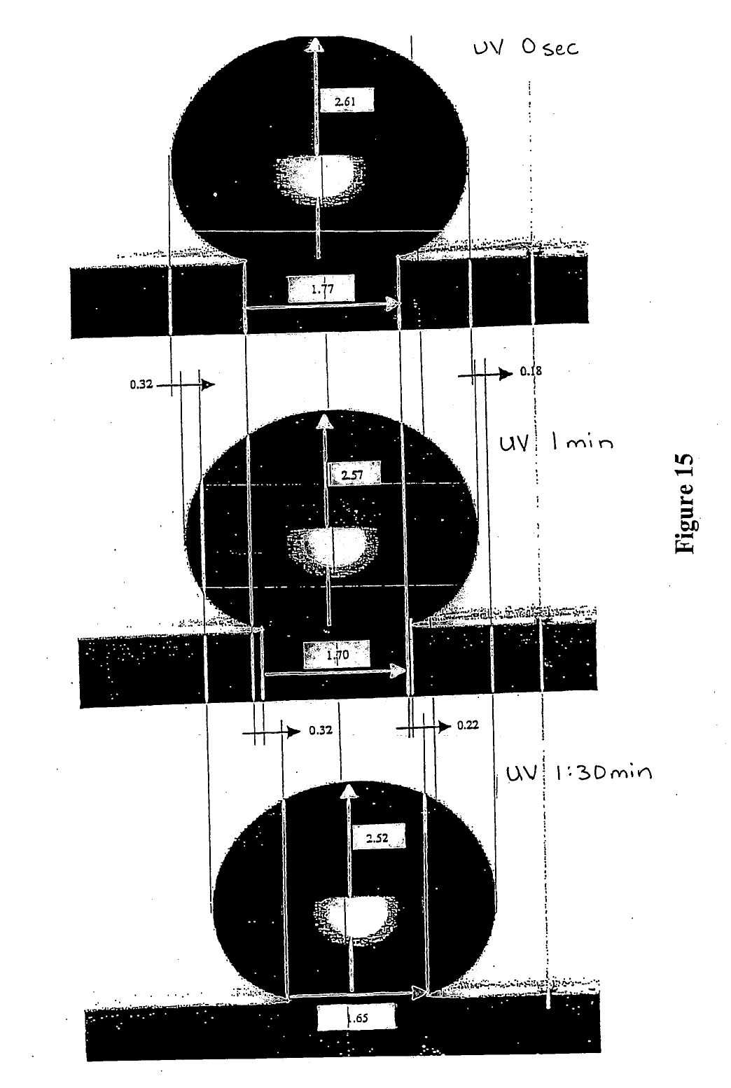

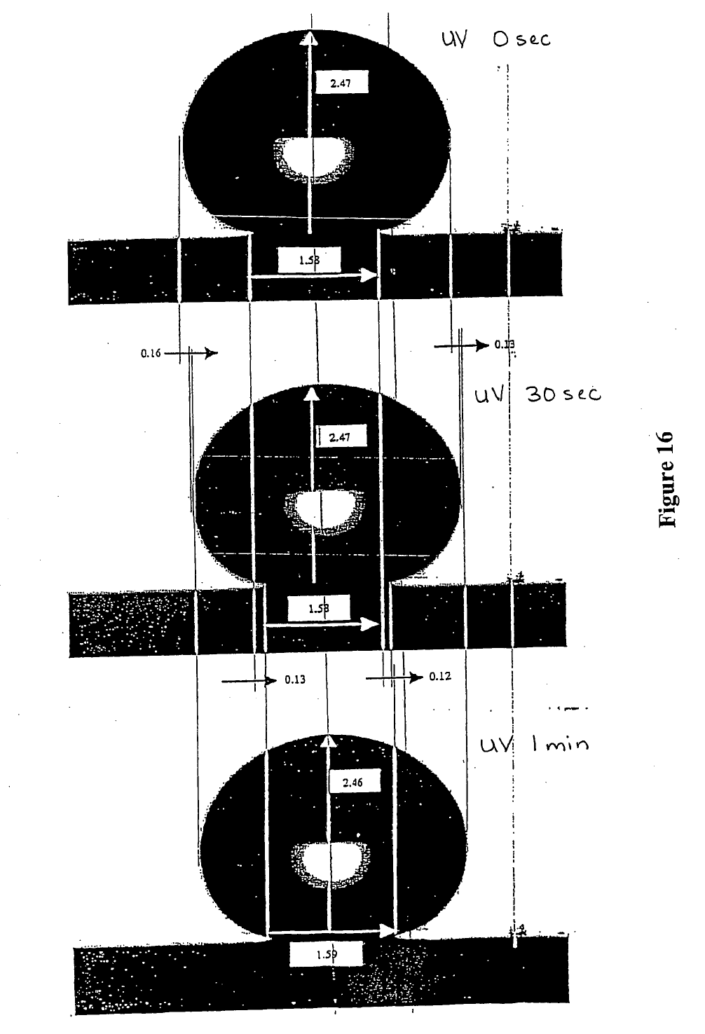

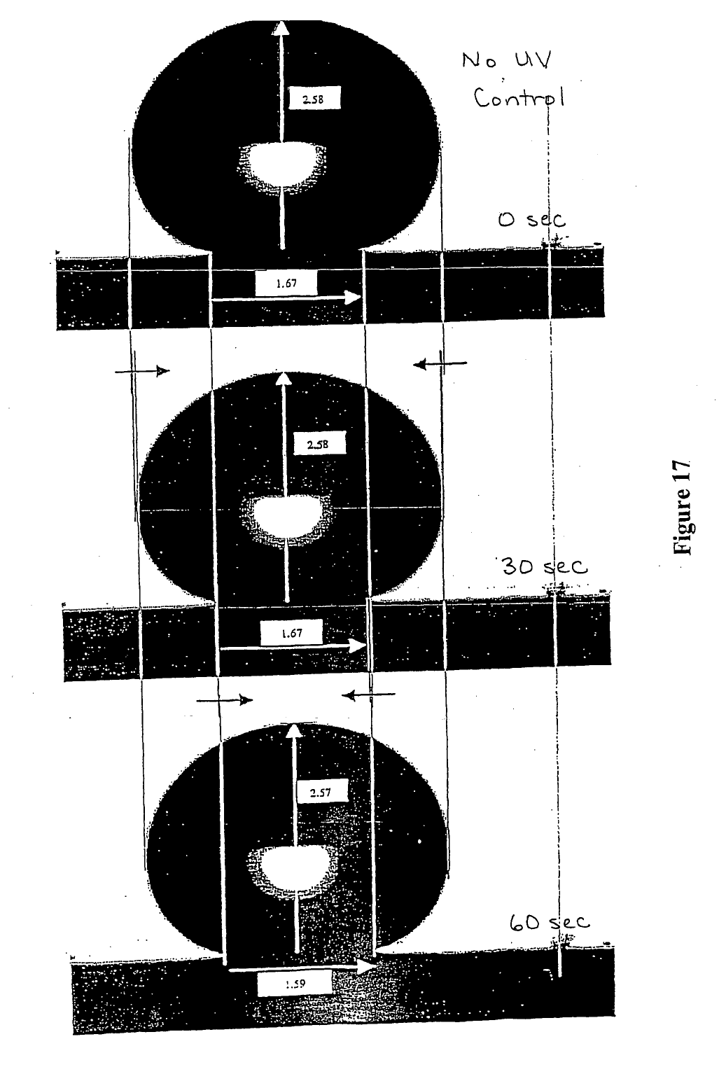

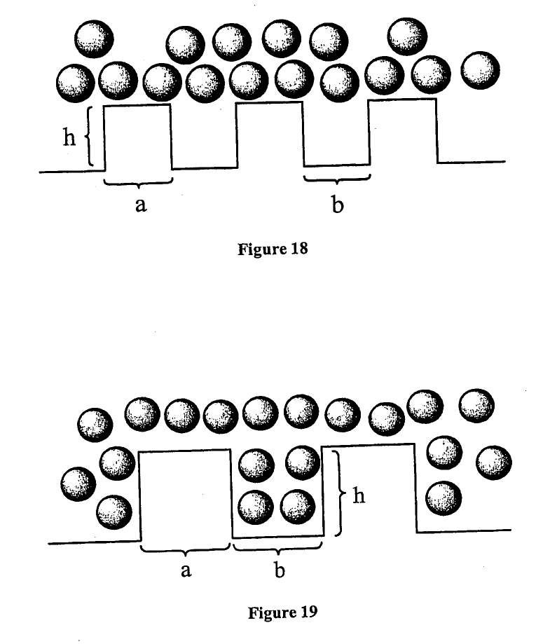

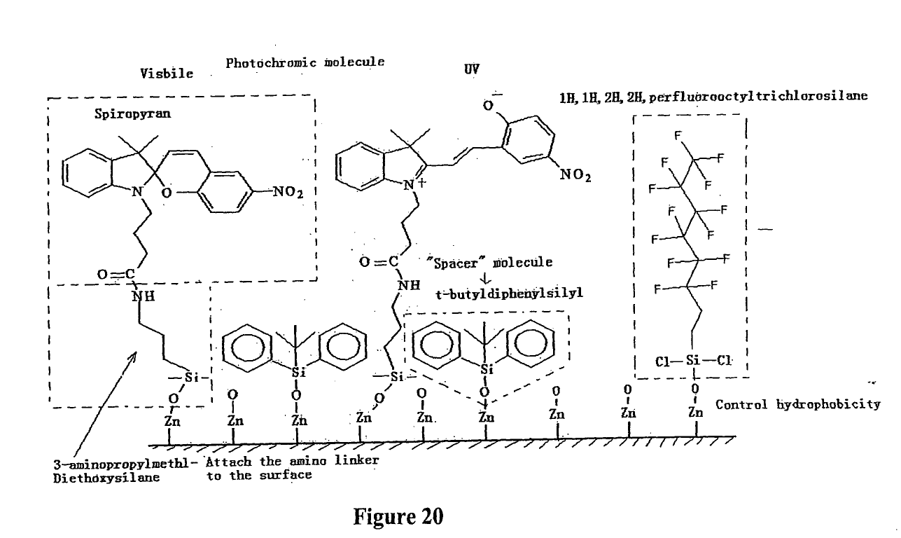

The surface chemistry of the samples was modified in two ways. Oxide-coated silicon nanowires bearing a superhydrophobic surface due to modification with perfluorooctyltrichlorosilane and tert-butyldiphenylchlorosilane gave contact angles near 180°. Smooth surfaces modified in this way gave lower contact angles. If such surfaces were subjected to UV-ozone treatment for a short time, the hydrophobicity was decreased slightly, which can be due to partial removal of the fluorocarbon silane coating. This change was observed via contact angle measurement. By progressive exposure to the UV-ozone treatment, smooth and nanowire samples of decreasing hydrophobicity can be prepared. The results are plotted graphically in FIG. 11. The vertical axis represents the contact angle on the nanowire surfaces (advancing or receding), and the horizontal axis represents the advancing contact angle on another region of the same sample having a smooth surface, and chemically treated along with the nanowire region.

In a second approach, smooth and nanowire surfaces were derivatized with the spiropyran. In this case, the surfaces can be reversibly switched from hydrophobic to more hydrophilic behavior with ultraviolet light, and then back to hydrophobic with visible light. After the surface treatment, the advancing and receding contact angles were determined for all samples. These are listed in Table 1 along with advancing and receding contact angle values corresponding to two of the data points in FIG. 11 (Samples 3 and 4 in Table 1).

### f) Theoretical Interpretation

As set forth above, Equation 9 extends the Cassie-Baxter treatment to heterogeneous fractal surfaces. The solid line in FIG. 11 is a fit of Eqn. 9 to the advancing contact angle data. Scanning electron microscope images and the standard box counting method were used to determine the fractal length scales and dimension of the nanowire surface: L=361 nm, l=40 nm, and D=2.48. The fit gave a value of 0.25 for f, which is the only adjustable parameter. The dashed line in FIG. 11 is a plot of the same equation with the same fractal parameters and f=1.0. In this limit, Eqn. 9 reduces to the Wenzel equation for fractal surfaces.

It is clear that a subset of the data for advancing contact angles is described by Eqn. 9 with f=0.25 (Cassie-Baxter wetting). Within this subset of the data, the fraction of the projected surface wetted by water is substantially constant regardless of the intrinsic contact angle measured on a smooth surface with the same chemical composition. The subset of advancing angle data that does not follow the Cassie-Baxter curve (near θ_{S,a}=90°) illustrates the point that for the multidimensional roughness exhibited by this surface, the contact angle depends upon its “history.” Interestingly, this second subset of points falls near the Wenzel curve, indicating that in these experiments, the drop can achieve Wenzel wetting, due to surface defects or vibration. The advancing angle is non-ergodic, and that advancing contact angles on rough surfaces may “fall” into the Wenzel case if the drop is moved slightly after application to the surface. See Herminghaus, S. Europhysics Letters 2000, 52, 165-170. The physical basis for this phenomenon is that surfaces with roughness at two or more different length scales can exclude liquid from an indentation due to smaller scale indentations along the ridge of the defect. The constant value of the fraction wetted is also a result of this multidimensional roughness since the excluded liquid would not fall into the indentation, and hence increase f dramatically to the value of 1 (i.e., Wenzel wetting), until the inherent contact angle became sufficiently low. In a similar way, while some of the receding contact angle data appears to be well described by eqn. 6 with f=1 (Wenzel wetting) another subset of the data is not in agreement with this interpretation (again near θ_{Σ,a}=90°).

Table 1 lists experimental roughness coefficients r that were obtained using scanning electron microscope images and the standard box counting method to determine the fractal length scales and dimensions of the different rough nanowire samples. Using the experimental smooth surface advancing/receding contact angles and the rough surface Wenzel advancing/receding contact angle data in Table 1, the factor 0.97 in eq. 8 was derived, as discussed above. As shown in Table 1, this empirical equation yields calculated roughness values with errors less than 9% from the measured roughness. See Kamusewitz, H.; Possart, W. App. Phys. A—Mat. Sci. Proc. 2003, 76, 899-902. Also, using the measured roughness value obtained from the fractal analysis yields the predicted θ_{W,r} values which agree closely with the observed values (Table 1).

The relationship between smooth contact angles and rough angles can predict the driving force for drop motion using light as discussed above. The driving force per unit width for light-induced droplet motion, F_{d}, is given by

F_{d}=γₗᵥ[cos(θₐ^{UV})−cos(θᵣ^{VIS})] (Eqn. 18)

See Oh, S.-K.; Nakagawa, M.; Ichimura, K. J. Mater. Chem. 2002, 12, 2262-2269.

On a rough surface, assuming that the droplet follows the Wenzel form of wetting, it can be shown that from Eqs. 17 and 18

$${F}_{d} = {( \frac{L}{l} )}^{D - 2} {γ}_{lv} [ cos ( {θ}_{S , a}^{UV} ) + cos ( {θ}_{S , a}^{VIS} ) - 2 cos ( 0.97 {θ}_{S , a}^{VIS} ) ] \qquad ( Eqn . 19 )$$

The driving force is directly proportional to roughness, r=(L/l)^{D-2} and it is increased by a more hydrophobic surface. Equation 19 may be used to guide surface design by showing the relative effects of surface geometry and chemistry on the driving force. However, dynamic angles may differ from static values and thus this equation may not directly predict the drop driving force once a drop is in motion.

Making a surface inherently more hydrophobic can favor drop motion primarily because the contact angle hysteresis diminishes as contact angles approach 180°. See Johnson, R. E.; Dettre, R. H. Advances in Chemistry 1964, 43, 112-135; Shirtcliffe, N. J.; McHale, G.; Newton, M. I.; Perry, C. C. Langmuir 2005, 21, 937-943. However, increased hydrophobicity can make it more difficult to place the drop in Wenzel wetting. Thus, from a design perspective there appears to be a trade-off with respect to surface hydrophobicity when moving droplets of water using light. In the case of large drops, hydrostatic pressure can force the drop into the Wenzel wetting state. Any anisotropic pressure can be used in place of hydrostatic pressure so that smaller drop sizes can be used. For example, acoustic methods for determining equilibrium contact angles can be adapted to force drops into the Wenzel wetting state, as could other mechanical means whereby drops are pushed down and then withdrawn in order to capture them in the receding state which favors Wenzel wetting. See Lam, C. N. C.; Wu, R.; Li, D.; Hair, J. L.; Neumann, A. W. Adv. Coll. Interface Sci. 2002, 96, 191.

TABLE 1
Experimental and calculated contact angles and surface roughness

| | Advancing/ | Exp. θ_{S} | Exp. θ_{W} | Exp. | | |
|---|---|---|---|---|---|---|
| | receding | smooth | rough | measured | | Calcd. θ_{W} for |
| | contact | surface | surface | roughnes | Calcd. r | rough surface |
| | angle | (deg) | (deg) | s (r) | (eq. 8) | (deg), (eq. 8) |
| Sample 1: Treated | Advancing | 110 +/− 2 | 132 +/− 2 | | | |
| with organosilanes | Receding | 74 +/− 2 | 113 +/− 4 | 1.76 | 1.90 | 118 |
| and photochrome, | | | | | | |
| under UV light | | | | | | |
| Sample 2: Treated | Advancing | 122 +/− 3 | 157 +/− 2 | | | |
| with organosilanes | Receding | 84 +/− 4 | 140 +/− 5 | 1.76 | 1.89 | 141 |
| Sample 3: Treated | Advancing | 113 +/− 3 | 170 +/− 2 | | | |
| with organosilanes | Receding | 80 +/− 3 | 159 +/− 1 | 2.87 | 2.71 | 156 |
| Sample 4: Treated | Advancing | 116 +/− 5 | 171 +/− 1 | | | |
| with organosilanes | Receding | 68 +/− 6 | 164 +/− 2 | 2.87 | 2.64 | 150 |

### g) Conclusions

When randomly patterned nanowires are combined with very hydrophobic surface coatings, water contact angles can be either in the Cassie-Baxter or Wenzel wetting modes depending upon the history of how they are applied to this fractally rough surface. In Cassie-Baxter wetting, the fraction of the projected surface wetted by water can be constant regardless of the intrinsic contact angle measured on a smooth surface with the same chemical composition. This observation is in line with Herminghaus' theory and observations for plant leaves with multidimensional roughness.

Using the notion of contact line energy, the driving force for light-induced water drop movement on superhydrophobic, light-responsive surfaces can be modeled as proportional to surface roughness and dependent upon the intrinsic hydrophobicity as well as the light-induced contact angle change on a similarly coated smooth surface. Since Wenzel wetting is the desired condition for water drop movement, increasing the size of the drop is a simple way to generate the additional downward pressure needed to force the drop into this configuration. By combining phenomenological equations and empirical observations, an equation predicting the driving force for light-induced movement of water drops in terms of fractal surface geometry and surface energy measured by advancing and receding contact angles on a similar smooth surface was developed. Although this equation may not directly predict the resultant driving force because dynamic contact angles may differ from static values, it can be used to gauge the relative effects of varying surface properties on the driving force for water drop movement using light.

### H. References

1. Abbott, S.; Ralston, J.; Reynolds, G.; Hayes, R. Langmuir 1999, 15, 8923-8938.
2. Adam, N. K.; Jessop, G. J. Chem. Soc. 1925, 127, 1863-1868.
3. Barthlott, W.; Neinhuis, C. Planta 1997, 202, 1-8.
4. Bico, J., Marzolin, C. & Quere, D. Pearl drops. Europhysics Letters 47, 220-226 (1999).
5. Blossey, R. Nature Materials 2003, 2, 301-306.
6. Blossey, R.; Bosio, A. Langmuir 2002, 18, 2952-2954.
7. Bunker, B. C.; Kim, B. I.; Houston, J. E.; Rosario, R.; Garcia, A. A.; Hayes, M.; Gust, D.; Picraux, S. T. Nano Letters 2003, 3, 1723-1727.
8. Cassie, A. B. & Baxter, S., Wettability of porous surfaces, Transactions of the Faraday Society 40, 546-551 (1944).
9. Chibowski, E., Surface free energy of a solid from contact angle hysteresis. Advances in Colloid and Interface Science 103, 149-172 (2003).
10. Darhuber, A. A.; Troian, S. M.; Reisner, W. W. Phys. Rev. E. 2001, 64, 316-333.
11. Deegan, R. D. Nature 1997, 389, 827-829.
12. Della Volpe, C.; Maniglio, D.; Morra, M.; Siboni, S. Colloids and Surfaces A 2002, 206, 47-67.
13. Garcia, A. A.; Cheman, S.; Park, J.; Gust, D.; Jahnke, F.; Rosario, R. J. Phys. Chem. A 2000, 104, 6103-6107.
14. Gaydos, J.; Neumann, A. W. Adv. Coll. Interface Sci. 1994, 49, 197-248.
15. Good, R. J. J. Am. Chem. Soc. 1952, 74, 5041-5042.
16. Grunze, M. Science 1999, 283, 41-42.
17. Hazlett, R. D. J. Colloid Interface Sci. 1990, 137, 527-533.
18. He, B., Patankar, N. A. & Lee, J., Multiple equilibrium droplet shapes and design criterion for rough hydrophobic surfaces, Langmuir 19, 4999-5003 (2003).
19. Herminghaus, S. Advanced Materials 1999, 11, 1393-1395.
20. Herminghaus, S. Europhysics Letters 2000, 52, 165-170.
21. Ichimura, K., Oh, S. & Nakagawa, M., Light-driven motion of liquids on a photoresponsive surface, Science 288, 1624-1626 (2000).
22. Ishihara, K.; Hamada, N.; Kato, S.; Shinohara, I. J. Polymer Sci., Polymer Chem. Ed. 1983, 21, 1551-1555.
23. Johnson, R. E.; Dettre, R. H. Advances in Chemistry 1964, 43, 112-135.
24. Johnson, R. E.; Dettre, R. H. J. Phys. Chem. 1964, 68, 1744-1750.
25. Kako, T.; Nakajima, A.; Irie, H.; Kato, Z.; Uematsu, K.; Watanabe, T.; Hashimoto, K. J. Mat. Sci. 2004, 39, 547-555.
26. Kamusewitz, H.; Possart, W. App. Phys. A—Mat. Sci. Proc. 2003, 76, 899-902.
27. Lafitma, A. & Quere, D., Superhydrophobic states. Nature Materials 2, 457-460 (2003).
28. Lahann, J. et al. A reversibly switching surface. Science 299, 371-374 (2003).
29. Lam, C. N. C.; Wu, R.; Li, D.; Hair, J. L.; Neumann, A. W. Adv. Coll. Interface Sci. 2002, 96, 191.
30. Lam, P.; Wynne, K. J.; Wnek, G. E. Langmuir 2002, 18, 948-951.
31. Li, D.; Neumann, A. W. Colloid. Polym. Sci. 1992, 270, 4989-504.
32. Liang, L., Ski, M., Viswanathan, V. V., Peummg, L. M. & Young, J. S., Temperature-sensitive polypropylene membranes prepared by plasma polymerization, Journal of Membrane Science 177, 97-108 (2000).
33. Marmur, A. Langrnuir 2004, 20, 3517-3519.
34. Nakajima, A.; Hashimoto, K.; Watanabe, T.; Takai, K.; Yamauchi, G.; Fujishima, A. Langmuir 2000, 16, 7044-7074.
35. Oh, S.-K.; Nakagawa, M.; Ichimura, K. J. Mater. Chem. 2002, 12, 2262-2269.
36. Onda, T., Shibuichi, S., Satoh, N. & Tsujii, K., Super-water-repellent fractal surfaces, Langmuir 12, 2125-2127 (1996).
37. Otten, A.; Herminghaus, S. Langmuir 2004, 20, 2405-2408.
38. Parker, A. R.; Lawrence, C. R. Nature 2001, 414, 33-34.
39. Patankar, N. A., On the modeling of hydrophobic contact angles on rough surfaces, Langmuir 19, 1249-1253 (2003).
40. Ranos, S. M. M., Charlaix, E. & Benyagoub, A. Contact angle hysteresis on nano-structured surfaces, Surface Science 540, 355-362 (2003).
41. Rosario, R. et al., Photon-modulated wettability changes on spiropyran-coated surfaces, Langmuir 18, 8062-8069 (2002).
42. Rosario, R.; Gust, D.; Garcia, A. A.; Hayes, M.; Taraci, J. L.; Clement, T.; Dailey, J. W.; Picraux, S. T. J. Phys. Chem. B 2004, 108, 12640-12642.
43. Rosario, R.; Gust, D.; Hayes, M.; Springer, J.; Garcia, A. A. Langmuir 2003, 19, 8801-8806.
44. Schneemilch, M., Welters, W. J., Hayes, R. A. & Ralston, J., Electrically induced changes in dynamic wettability, Langmuir 16, 2924-2927 (2000).
45. Shibuichi, S., Yamamoto, T., Onda, T. & Tsujii, K., Super water- and oil-repellent surfaces resulting from fractal structure, Journal of Colloid and Interface Science 208, 287-294 (1998).
46. Shin, J.-Y., Kuo, C.-W., Chert, P. & Moth C.-Y. Fabrication of tunable superhydrophobic surfaces by nanosphere lithography, Chemistry of Materials 16, 561-564 (2004).
47. Shirtcliffe, N. J.; McHale, G.; Newton, M. I.; Perry, C. C. Langmuir 2005, 21, 937-943.
48. Tadmore, R. Langmuir 2004, 20, 7659-7664.
49. Vicsec, T., Fractal growth phenomena (World Scientific, Singapore, 1989).
50. von Baeyer, H. C. The Sciences: J. New York Acad. Sci. 2000, 2000, 12-15.
51. Wagner, R. S. in Whisker Technology (ed. Levit, A. P.) 47-119 (Wiley-Interscience, New York, 1970).
52. Wang, R. Nature 1997, 388, 431-432.
53. Washizu, M. IEEE Trans. Ind. Appl. 1998, 34, 732-737.
54. Wenzel, R. N., Resistance of solid surfaces to wetting by water, Industrial and Engineering Chemistry Research 28, 988-994 (1936).
55. Yakushiji, T. & Sakai, K., Graft architectural effects on thermoresponsive wettability changes of poly(N-isopropylacrylamide)-modified surfaces, Langmuir 14, 4657-4662 (1998).
56. Zhao, B.; Moore, J. S.; Beebe, D. J. Anal. Chem. 2002, 74, 4259-4268.

1. A device comprising a surface, wherein the surface has roughness, a hydrophobic layer, and a photoresponsive molecule.

2. The device of claim 1, wherein the roughness is either a well ordered microstructure or a random fractal geometry.

3. (canceled)

4. The device of claim 1, wherein the device comprises a rough fractal surface.

5-6. (canceled)

7. The device of claim 2, wherein the random fractal geometry comprises at least one a nanowire.

8. The device of claim 4, wherein the nanowire is a silicon, zinc, silicon dioxide, titanium, tungsten, tantalum, iron, nickel, or alloy nanowire.

9. (canceled)

10. The device of claim 4, wherein the nanowire is in a random array of nanowires, an ordered array of nanowires, a hierarchically patterned array of nanowires, or in a combination of each.

11-14. (canceled)

15. The device of claim 1, wherein the photoresponsive molecule is an isomerization molecule which can be isomerized into a first and a second form, wherein the first and second forms have different effects on the wetting of the surface by a fluid.

16-20. (canceled)

21. The device of claim 15, wherein the photoresponsive molecule is a spiropyran.

22. The device of claim 21, wherein the spiropyran is an indolinospiropyran.

23. The device of claim 15, wherein the photoresponsive molecule is a spirooxazine, benzo-naphthopyran, naphthopyran, azobenzene, fulgide, diarylethene, dihydroindolizine, photochromic quinone, perimidinespirocyclohexadienone, or dihydropyrene or combinations thereof.

24. (canceled)

25. The device of claim 1, further comprising a liquid droplet, wherein the liquid droplet has a contact angle with the surface, and wherein the contact angle is magnified relative to a smooth surface and wherein the contact angle hysteresis is lowered relative to a smooth surface.

26. The device of claim 1, wherein the surface is a superhydrophobic surface.

27. The device of claim 1, further comprising a liquid droplet, wherein the liquid droplet has a contact angle with the surface, and wherein the advancing contact angle under a first condition is lower than the receding contact angle under a second condition.

28-30. (canceled)

31. The device of claim 13, wherein the difference in the contact angle hysteresis as defined by the advancing contact angle and the receding contact angle is less than the difference in the contact angle determined by two different light conditions.

32-43. (canceled)

44. The device of claim 1, wherein contact angle switching is amplified and wherein contact angle hysteresis is decreased.

45-65. (canceled)

66. A method of inducing linear movement of a fluid droplet on a surface comprising the steps of: a) providing a rough surface coated with a photosensitive variable hydrophobicity agent; b) positioning a fluid droplet in contact with the surface; and c) exposing the surface and droplet to a variable wavelength light gradient.

67. The method of claim 66, wherein the providing a rough surface step comprises the steps of: a) providing a polished silicon surface; and b) increasing the roughness of the surface by vapor deposition of silicon, thereby providing a surface having a three dimensional fractal dimension of at least about 2.2.

68. (canceled)

69. The method of claim 66, wherein coating the surface with a photosensitive variable hydrophobicity agent comprises the steps of a) treating the surface with tert-butyldiphenylchlorosilane; b) treating the surface with perfluorooctyltrichlorosilane; c) treating the surface with 3-aminopropyldiethoxymethylsilane; and d) treating the surface with a photosensitive variable hydrophobicity agent.

70. A method of preparing a surface comprising the steps of: a) providing a surface having a three dimensional fractal dimension of at least about 2.2; and b) coating the surface with a photosensitive variable hydrophobicity agent.

71. The method of claim 70, wherein the surface is prepared by vapor deposition.

72. The method of claim 70, wherein the surface comprises silicon, zinc, silicon dioxide, titanium, tungsten, tantalum, iron, nickel, or compounds, mixtures, or alloys thereof.

73. The method of claim 70, wherein the providing step comprises a) treating the surface with a branched organic halosilane and/or a fluorinated halosilane; b) binding a photosensitive variable hydrophobicity agent to the surface.

74. The method of claim 70, wherein the photosensitive variable hydrophobicity agent is a spiropyran.

75. The device of claim 1, wherein the surface is a hydrophobic surface.
